SEQUENCE LISTING (37 sequences)

SEQ ID NO: 1 - DNA, 20 bp, Artificial Sequence
  Primer M&M Standard PCR of Hobit
caccgctgcagaacagaaag

SEQ ID NO: 2 - DNA, 17 bp, Artificial Sequence
  M&M Primer Standard PCR of Hobit 2.0
gcaagagcagtgagctg

SEQ ID NO: 3 - DNA, 25 bp, Artificial Sequence
  Primer Hobit-L 4.0 CLN
ggtacctcgccctggaggtacaggg

SEQ ID NO: 4 - DNA, 17 bp, Artificial Sequence
  Primer Hobit-L 2.0 CLN
gcaagagcagtgagctg

SEQ ID NO: 5 - DNA, 21 bp, Artificial Sequence
  Primer Lentiviral Vector Construction and Infection Hobit RT 9.0
catatgtggcaagagctttgg

SEQ ID NO: 6 - DNA, 20 bp, Artificial Sequence
  Lentiviral Vector Construction and Infection Primer Hobit RT 9.0
ggcaagttgagtgaagctct

SEQ ID NO: 7 - protein, 23 aa, Homo sapiens
YECNVCAKTFGQLSNLKVHLRVH

SEQ ID NO: 8 - protein, 23 aa, Homo sapiens
YECNICGKSFGQLSNLKVHLRVH

SEQ ID NO: 9 - protein, 23 aa, Homo sapiens
HECQVCHKRFSSTSNLKTHLRLH

SEQ ID NO: 10 - protein, 23 aa, Homo sapiens
REREVCHKRFSSSSNLKTHLRLH

SEQ ID NO: 11 - protein, 23 aa, Homo sapiens
FKCQTCNKGFTQLAHLQKHYLVH

SEQ ID NO: 12 - protein, 23 aa, Homo sapiens
FQCALCQKSFTQLAHLQKHHLVH

SEQ ID NO: 13 - protein, 23 aa, Homo sapiens
YQCKVCPAKFTQFVHLKLHKRLH

SEQ ID NO: 14 - protein, 23 aa, Homo sapiens
FQCSVCRSRFTQHIHLKLHHRLH

SEQ ID NO: 15 - protein, 23 aa, Artificial sequence
  Consensus sequence zinc finger Hobit
FECNVCXKRFTQLSNLKLHLRLH

SEQ ID NO: 16 - DNA, 1530 bp, Homo sapiens
  CDS (1)..(1530)
atggccctgggaggtacagggggctccctgtcccccagcctggacttccagctcttccgaggtgaccaggtcttctcagcctgcagaccacttccagacatggtggatgctcatggcccatcctgtgccagctggctgtgtcccttgcccctggcaccgggcaggtctgcactgctggcctgcctacaggacctggacctgaacctgtgcaccccacagccggcacccctgggcacagacctgcagggcctccaagaggacgccttgagcatgaagcacgagccaccagggctgcaggccagctccaccgatgacaagaaattcacagtcaagtacccacagaacaaggacaagctgggaaaacagccagaaagagctggcgagggggccccctgcccagccttctcctctcataacagctcttccccaccaccgctgcagaacagaaagagccccagccccttggctttctgcccctgtccccctgtcaactccatctccaaggagctcccatttctcctccacgccttctaccctggatacccacttctcctgcctccacccaacctgttcacctatggggccctaccttctgaccaatgtccccacctcctcatgctgccccaagacccctcctaccccaccatggctatgcctagcctgctgatgatggtcaatgagctggggcaccccagcgctcggtgggagaccctgcttccctacccaggggccttccaagcctctggccaagctctgccttcccaggcccgaaatccaggtgctggagctgccccaaccgactccccaggcctggagcgtggtggcatggcatctccagcaaagcgggtcccattgagttcccagacaggcaccgcagccttgccttacccgctgaaaaagaagaatggcaaaatcctgtacgagtgcaacatatgtggcaagagctttgggcagctctccaatctcaaggtccacctgcgtgtgcacagtggagagcgtccattccagtgtgccttgtgccagaagagcttcactcaacttgcccacctgcagaagcaccacctggtgcacactggggagcggccccacaagtgctcgataccgtgggttccaggaagaaatcactggaaaagcttccaggcctggagggaaagagaggtgtgccacaagcgcttcagcagctccagtaacctcaagacccacctgcgcctgcactccggggcccggcccttccagtgcagtgtctgccggagtcgcttcacccagcacatccacctgaagctgcaccatcggctgcatgccccacagccctgtggcctggtgcacacccagctgcccctggcctctctggcctgccttgcccaatggcaccagggggcactagatcttatggcggtggcatctgagaaacacatgggctatgacatagatgaggtcaaagtgtcctcgacatcccaggggaaagcaagagcagtgagcctgagcagtgccgggactcccctggtgatggggcaggaccagaacaattaa

SEQ ID NO: 17 - protein, 509 aa, Homo sapiens
MALGGTGGSLSPSLDFQLFRGDQVFSACRPLPDMVDAHGPSCASWLCPLPLAPGRSALLACLQDLDLNLCTPQPAPLGTDLQGLQEDALSMKHEPPGLQASSTDDKKFTVKYPQNKDKLGKQPERAGEGAPCPAFSSHNSSSPPPLQNRKSPSPLAFCPCPPVNSISKELPFLLHAFYPGYPLLLPPPNLFTYGALPSDQCPHLLMLPQDPSYPTMAMPSLLMMVNELGHPSARWETLLPYPGAFQASGQALPSQARNPGAGAAPTDSPGLERGGMASPAKRVPLSSQTGTAALPYPLKKKNGKILYECNICGKSFGQLSNLKVHLRVHSGERPFQCALCQKSFTQLAHLQKHHLVHTGERPHKCSIPWVPGRNHWKSFQAWREREVCHKRFSSSSNLKTHLRLHSGARPFQCSVCRSRFTQHIHLKLHHRLHAPQPCGLVHTQLPLASLACLAQWHQGALDLMAVASEKHMGYDIDEVKVSSTSQGKARAVSLSSAGTPLVMGQDQNN

SEQ ID NO: 18 - DNA, 1530 bp, Homo sapiens; identical to SEQ ID NO: 16
  CDS (1)..(1530)

SEQ ID NO: 19 - protein, 509 aa, Homo sapiens; identical to SEQ ID NO: 17

SEQ ID NO: 20 - DNA, 1470 bp, Homo sapiens
  CDS (1)..(1470)
atggccctgggaggtacagggggctccctgtcccccagcctggacttccagctcttccgaggtgaccaggtcttctcagcctgcagaccacttccagacatggtggatgctcatggcccatcctgtgccagctggctgtgtcccttgcccctggcaccgggcaggtctgcactgctggcctgcctacaggacctggacctgaacctgtgcaccccacagccggcacccctgggcacagacctgcagggcctccaagaggacgccttgagcatgaagcacgagccaccagggctgcaggccagctccaccgatgacaagaaattcacagtcaagtacccacagaacaaggacaagctgggaaaacagccagaaagagctggcgagggggccccctgcccagccttctcctctcataacagctcttccccaccaccgctgcagaacagaaagagccccagccccttggctttctgcccctgtccccctgtcaactccatctccaaggagctcccatttctcctccacgccttctaccctggatacccacttctcctgcctccacccaacctgttcacctatggggccctaccttctgaccaatgtccccacctcctcatgctgccccaagacccctcctaccccaccatggctatgcctagcctgctgatgatggtcaatgagctggggcaccccagcgctcggtgggagaccctgcttccctacccaggggccttccaagcctctggccaagctctgccttcccaggcccgaaatccaggtgctggagctgccccaaccgactccccaggcctggagcgtggtggcatggcatctccagcaaagcgggtcccattgagttcccagacaggcaccgcagccttgccttacccgctgaaaaagaagaatggcaaaatcctgtacgagtgcaacatatgtggcaagagctttgggcagctctccaatctcaaggtccacctgcgtgtgcacagtggagagcgtccattccagtgtgccttgtgccagaagagcttcactcaacttgcccacctgcagaagcaccacctggtgcacactggggagcggccccacaagtgctcggtgtgccacaagcgcttcagcagctccagtaacctcaagacccacctgcgcctgcactccggggcccggcccttccagtgcagtgtctgccggagtcgcttcacccagcacatccacctgaagctgcaccatcggctgcatgccccacagccctgtggcctggtgcacacccagctgcccctggcctctctggcctgccttgcccaatggcaccagggggcactagatcttatggcggtggcatctgagaaacacatgggctatgacatagatgaggtcaaagtgtcctcgacatcccaggggaaagcaagagcagtgagcctgagcagtgccgggactcccctggtgatggggcaggaccagaacaattaa

SEQ ID NO: 21 - protein, 489 aa, Homo sapiens
MALGGTGGSLSPSLDFQLFRGDQVFSACRPLPDMVDAHGPSCASWLCPLPLAPGRSALLACLQDLDLNLCTPQPAPLGTDLQGLQEDALSMKHEPPGLQASSTDDKKFTVKYPQNKDKLGKQPERAGEGAPCPAFSSHNSSSPPPLQNRKSPSPLAFCPCPPVNSISKELPFLLHAFYPGYPLLLPPPNLFTYGALPSDQCPHLLMLPQDPSYPTMAMPSLLMMVNELGHPSARWETLLPYPGAFQASGQALPSQARNPGAGAAPTDSPGLERGGMASPAKRVPLSSQTGTAALPYPLKKKNGKILYECNICGKSFGQLSNLKVHLRVHSGERPFQCALCQKSFTQLAHLQKHHLVHTGERPHKCSVCHKRFSSSSNLKTHLRLHSGARPFQCSVCRSRFTQHIHLKLHHRLHAPQPCGLVHTQLPLASLACLAQWHQGALDLMAVASEKHMGYDIDEVKVSSTSQGKARAVSLSSAGTPLVMGQDQNN

SEQ ID NO: 22 - DNA, 1404 bp, Homo sapiens
  CDS (1)..(1404)
atggccctgggaggtacagggggctccctgtcccccagcctggacttccagctcttccgaggtgaccaggtcttctcagcctgcagaccacttccagacatggtggatgctcatggcccatcctgtgccagctggctgtgtcccttgcccctggcaccgggcaggtctgcactgctggcctgcctacaggacctggacctgaacctgtgcaccccacagccggcacccctgggcacagacctgcagggcctccaagaggacgccttgagcatgaagcacgagccaccagggctgcaggccagctccaccgatgacaagaaattcacagtcaagtacccacagaacaaggacaagctgggaaaacagccagaaagagctggcgagggggccccctgcccagccttctcctctcataacagctcttccccaccaccgctgcagaacagaaagagccccagccccttggctttctgcccctgtccccctgtcaactccatctccaaggagctcccatttctcctccacgccttctaccctggatacccacttctcctgcctccacccaacctgttcacctatggggccctaccttctgaccaatgtccccacctcctcatgctgccccaagacccctcctaccccaccatggctatgcctagcctgctgatgatggtcaatgagctggggcaccccagcgctcggtgggagaccctgcttccctacccaggggccttccaagcctctggccaagctctgccttcccaggcccgaaatccaggtgctggagctgccccaaccgactccccaggcctggagcgtggtggcatggcatctccagcaaagcgggtcccattgagttcccagacaggcaccgcagccttgccttacccgctgaaaaagaagaatggcaaaatcctgtacgagtgcaacatatgtggcaagagctttgggcagctctccaatctcaaggtccacctgcgtgtgcacagtggagagcgtccattccagtgtgccttgtgccagaagagcttcactcaacttgcccacctgcagaagcaccacctggtgcacactggggagcggccccacaagtgctcggtgtgccacaagcgcttcagcagctccagtaacctcaagacccacctgcgcctgcactccggggcccggcccttccagtgcagtgtctgccggagtcgcttcacccagctgcccctggcctctctggcctgccttgcccaatggcaccagggggcactagatcttatggcggtggcatctgagaaacacatgggctatgacatagatgaggtcaaagtgtcctcgacatcccaggggaaagcaagagcagtgagcctgagcagtgccgggactcccctggtgatggggcaggaccagaacaattaa

SEQ ID NO: 23 - protein, 467 aa, Homo sapiens
MALGGTGGSLSPSLDFQLFRGDQVFSACRPLPDMVDAHGPSCASWLCPLPLAPGRSALLACLQDLDLNLCTPQPAPLGTDLQGLQEDALSMKHEPPGLQASSTDDKKFTVKYPQNKDKLGKQPERAGEGAPCPAFSSHNSSSPPPLQNRKSPSPLAFCPCPPVNSISKELPFLLHAFYPGYPLLLPPPNLFTYGALPSDQCPHLLMLPQDPSYPTMAMPSLLMMVNELGHPSARWETLLPYPGAFQASGQALPSQARNPGAGAAPTDSPGLERGGMASPAKRVPLSSQTGTAALPYPLKKKNGKILYECNICGKSFGQLSNLKVHLRVHSGERPFQCALCQKSFTQLAHLQKHHLVHTGERPHKCSVCHKRFSSSSNLKTHLRLHSGARPFQCSVCRSRFTQLPLASLACLAQWHQGALDLMAVASEKHMGYDIDEVKVSSTSQGKARAVSLSSAGTPLVMGQDQNN

SEQ ID NO: 24 - DNA, 1110 bp, Homo sapiens
  CDS (1)..(1110)
atggccctgggaggtacagggggctccctgtcccccagcctggacttccagctcttccgaggtgaccaggtcttctcagcctgcagaccacttccagacatggtggatgctcatggcccatcctgtgccagctggctgtgtcccttgcccctggcaccgggcaggtctgcactgctggcctgcctacaggacctggacctgaacctgtgcaccccacagccggcacccctgggcacagacctgcagggcctccaagaggacgccttgagcatgaagcacgagccaccagggctgcaggccagctccaccgatgacaagaaattcacagtcaagtacccacagaacaaggacaagctgggaaaacagccagaaagagctggcgagggggccccctgcccagccttctcctctcataacagctcttccccaccaccgctgcagaacagaaagagccccagccccttggctttctgcccctgtccccctgtcaactccatctccaaggagctcccatttctcctccacgccttctaccctggatacccacttctcctgcctccacccaacctgttcacctatggggccctaccttctgaccaatgtccccacctcctcatgctgccccaagacccctcctaccccaccatggctatgcctagcctgctgatgatggtcaatgagctggggcaccccagcgctcggtgggagaccctgcttccctacccaggggccttccaagcctctggccaagctctgccttcccaggcccgaaatccaggtgctggagctgccccaaccgactccccaggcctggagcgtggtggcatggcatctccagcaaagcgggtcccattgagttcccagacaggcaccgcagccttgccttacccgctgaaaaagaagaatggcaaaatcctgtacgagtgcaacatatgtggcaagagctttgggcagctctccaatctcaagtggagagcgtccattccagtgtgccttgtgccagaagagcttcactcaacttgcccacctgcagaagcaccacctggtgcacactggggagcggccccacaagtgctcggtgtgccacaagcgcttcagcagctccagtaa

SEQ ID NO: 25 - protein, 369 aa, Homo sapiens
MALGGTGGSLSPSLDFQLFRGDQVFSACRPLPDMVDAHGPSCASWLCPLPLAPGRSALLACLQDLDLNLCTPQPAPLGTDLQGLQEDALSMKHEPPGLQASSTDDKKFTVKYPQNKDKLGKQPERAGEGAPCPAFSSHNSSSPPPLQNRKSPSPLAFCPCPPVNSISKELPFLLHAFYPGYPLLLPPPNLFTYGALPSDQCPHLLMLPQDPSYPTMAMPSLLMMVNELGHPSARWETLLPYPGAFQASGQALPSQARNPGAGAAPTDSPGLERGGMASPAKRVPLSSQTGTAALPYPLKKKNGKILYECNICGKSFGQLSNLKWRASIPVCLVPEELHSTCPPAEAPPGAHWGAAPQVLGVPQALQQLQ

SEQ ID NO: 26 - DNA, 903 bp, Homo sapiens
  CDS (1)..(903)
atggccctgggaggtacagggggctccctgtcccccagcctggacttccagctcttccgaggtgaccaggtcttctcagcctgcagaccacttccagacatggtggatgctcatggcccatcctgtgccagctggctgtgtcccttgcccctggcaccgggcaggtctgcactgctggcctgcctacaggacctggacctgaacctgtgcaccccacagccggcacccctgggcacagacctgcagggcctccaagaggacgccttgagcatgaagcacgagccaccagggctgcaggccagctccaccgatgacaagaaattcacagtcaagtacccacttctcctgcctccacccaacctgttcacctatggggccctaccttctgaccaatgtccccacctcctcatgctgccccaagacccctcctaccccaccatggctatgcctagcctgctgatgatggtcaatgagctggggcaccccagcgctcggtgggagaccctgcttccctacccaggggccttccaagcctctggccaagctctgccttcccaggcccgaaatccaggtgctggagctgccccaaccgactccccaggcctggagcgtggtggcatggcatctccagcaaagcgggtcccattgagttcccagacaggcaccgcagccttgccttacccgctgaaaaagaagaatggcaaaatcctgtacgagtgcaacatatgtggcaagagctttgggcagctctccaatctcaagtggagagcgtccattccagtgtgccttgtgccagaagagcttcactcaacttgcccacctgcagaagcaccacctggtgcacactggggagcggccccacaagtgctcggtgtgccacaagcgcttcagcagctccagtaa

SEQ ID NO: 27 - protein, 300 aa, Homo sapiens
MALGGTGGSLSPSLDFQLFRGDQVFSACRPLPDMVDAHGPSCASWLCPLPLAPGRSALLACLQDLDLNLCTPQPAPLGTDLQGLQEDALSMKHEPPGLQASSTDDKKFTVKYPLLLPPPNLFTYGALPSDQCPHLLMLPQDPSYPTMAMPSLLMMVNELGHPSARWETLLPYPGAFQASGQALPSQARNPGAGAAPTDSPGLERGGMASPAKRVPLSSQTGTAALPYPLKKKNGKILYECNICGKSFGQLSNLKWRASIPVCLVPEELHSTCPPAEAPPGAHWGAAPQVLGVPQALQQLQ

SEQ ID NO: 28 - DNA, 1725 bp, Homo sapiens
ggggaggtagcacaaagggaggaccatggtcttctaactgaggaacctgagcagatgccttgacccagaccagagacccaagccaaggaaagcccatgatcaccagagtaatggggatatgaaggaagaatcagctgcacaattaggttgttgtcataggcccatggccctgggaggtacagggggctccctgtcccccagcctggacttccagctcttccgaggtgaccaggtcttctcagcctgcagaccacttccagacatggtggatgctcatggcccatcctgtgccagctggctgtgtcccttgcccctggcaccgggcaggtctgcactgctggcctgcctacaggacctggacctgaacctgtgcaccccacagccggcacccctgggcacagacctgcagggcctccaagaggacgccttgagcatgaagcacgagccaccagggctgcaggccagctccaccgatgacaagaaattcacagtcaagtacccacagaacaaggacaagctgggaaaacagccagaaagagctggcgagggggccccctgcccagccttctcctctcataacagctcttccccaccaccgctgcagaacagaaagagccccagccccttggctttctgcccctgtccccctgtcaactccatctccaaggagctcccatttctcctccacgccttctaccctggatacccacttctcctgcctccacccaacctgttcacctatggggccctaccttctgaccaatgtccccacctcctcatgctgccccaagacccctcctaccccaccatggctatgcctagcctgctgatgatggtcaatgagctggggcaccccagcgctcggtgggagaccctgcttccctacccaggggccttccaagcctctggccaagctctgccttcccaggcccgaaatccaggtgctggagctgccccaaccgactccccaggcctggagcgtggtggcatggcatctccagcaaagcgggtcccattgagttcccagacaggcaccgcagccttgccttacccgctgaaaaagaagaatggcaaaatcctgtacgagtgcaacatatgtggcaagagctttgggcagctctccaatctcaaggtccacctgcgtgtgcacagtggagagcgtccattccagtgtgccttgtgccagaagagcttcactcaacttgcccacctgcagaagcaccacctggtgcacactggggagcggccccacaagtgctcgataccgtgggttccaggaagaaatcactggaaaagcttccaggcctggagggaaagagaggtgtgccacaagcgcttcagcagctccagtaacctcaagacccacctgcgcctgcactccggggcccggcccttccagtgcagtgtctgccggagtcgcttcacccagcacatccacctgaagctgcaccatcggctgcatgccccacagccctgtggcctggtgcacacccagctgcccctggcctctctggcctgccttgcccaatggcaccagggggcactagatcttatggcggtggcatctgagaaacacatgggctatgacatagatgaggtcaaagtgtcctcgacatcccaggggaaagcaagagcagtgagcctgagcagtgccgggactcccctggtgatggggcaggaccagaacaattaaaaatgttcttctgtcaaaaaaaaaaaaaaaat

SEQ ID NO: 29 - DNA, 1725 bp, Homo sapiens
ggggaggtagcacaaagggaggaccatggtcttctaactgaggaacctgagcagatgccttgacccagaccagagacccaagccaaggaaagcccatgatcaccagagtaatggggatatgaaggaagaatcagctgcacaattaggttgttgtcataggcccatggccctgggaggtacagggggctccctgtcccccagcctggacttccagctcttccgaggtgaccaggtcttctcagcctgcagaccacttccagacatggtggatgctcatggcccatcctgtgccagctggctgtgtcccttgcccctggcaccgggcaggtctgcactgctggcctgcctacaggacctggacctgaacctgtgcaccccacagccggcacccctgggcacagacctgcagggcctccaagaggacgccttgagcatgaagcacgagccaccagggctgcaggccagctccaccgatgacaagaaattcacagtcaagtacccacagaacaaggacaagctgggaaaacagccagaaagagctggcgagggggccccctgcccagccttctcctctcataacagctcttccccaccaccgctgcagaacagaaagagccccagccccttggctttctgcccctgtccccctgtcaactccatctccaaggagctcccatttctcctccacgccttctaccctggatacccacttctcctgcctccacccaacctgttcacctatggggccctaccttctgaccaatgtccccacctcctcatgctgccccaagacccctcctaccccaccatggctatgcctagcctgctgatgatggtcaatgagctggggcaccccagcgctcggtgggagaccctgcttccctacccaggggccttccaagcctctggccaagctctgccttcccaggcccgaaatccaggtgctggagctgccccaaccgactccccaggcctggagcgtggtggcatggcatctccagcaaagcgggtcccattgagttcccagacaggcaccgcagccttgccttacccgctgaaaaagaagaatggcaaaatcctgtacgagtgcaacatatgtggcaagagctttgggcagctctccaatctcaaggtccacctgcgtgtgcacagtggagagcgtccattccagtgtgccttgtgccagaagagcttcactcaacttgcccacctgcagaagcaccacctggtgcacactggggagcggccccacaagtgctcgataccgtgggttccaggaagaaatcactggaaaagcttccaggcctggagggaaagagaggtgtgccacaagcgcttcagcagctccagtaacctcaagacccacctgcgcctgcactccggggcccggcccttccagtgcagtgtctgccggagtcgcttcacccagcacatccacctgaagctgcaccatcggctgcatgccccacagccctgtggcctggtgcacacccagctgcccctggcctctctggcctgccttgcccaatggcaccagggggcactagatcttatggcggtggcatctgagaaacacatgggctatgacatagatgaggtcaaagtgtcctcgacatcccaggggaaagcaagagcagtgagcctgagcagtgccgggactcccctggtgatggggcaggaccagaacaattaaaaatgtttcttctgtcaaaaaaaaaaaaaaaa

SEQ ID NO: 30 - DNA, 1665 bp, Homo sapiens
ggggaggtagcacaaagggaggaccatggtcttctaactgaggaacctgagcagatgccttgacccagaccagagacccaagccaaggaaagcccatgatcaccagagtaatggggatatgaaggaagaatcagctgcacaattaggttgttgtcataggcccatggccctgggaggtacagggggctccctgtcccccagcctggacttccagctcttccgaggtgaccaggtcttctcagcctgcagaccacttccagacatggtggatgctcatggcccatcctgtgccagctggctgtgtcccttgcccctggcaccgggcaggtctgcactgctggcctgcctacaggacctggacctgaacctgtgcaccccacagccggcacccctgggcacagacctgcagggcctccaagaggacgccttgagcatgaagcacgagccaccagggctgcaggccagctccaccgatgacaagaaattcacagtcaagtacccacagaacaaggacaagctgggaaaacagccagaaagagctggcgagggggccccctgcccagccttctcctctcataacagctcttccccaccaccgctgcagaacagaaagagccccagccccttggctttctgcccctgtccccctgtcaactccatctccaaggagctcccatttctcctccacgccttctaccctggatacccacttctcctgcctccacccaacctgttcacctatggggccctaccttctgaccaatgtccccacctcctcatgctgccccaagacccctcctaccccaccatggctatgcctagcctgctgatgatggtcaatgagctggggcaccccagcgctcggtgggagaccctgcttccctacccaggggccttccaagcctctggccaagctctgccttcccaggcccgaaatccaggtgctggagctgccccaaccgactccccaggcctggagcgtggtggcatggcatctccagcaaagcgggtcccattgagttcccagacaggcaccgcagccttgccttacccgctgaaaaagaagaatggcaaaatcctgtacgagtgcaacatatgtggcaagagctttgggcagctctccaatctcaaggtccacctgcgtgtgcacagtggagagcgtccattccagtgtgccttgtgccagaagagcttcactcaacttgcccacctgcagaagcaccacctggtgcacactggggagcggccccacaagtgctcggtgtgccacaagcgcttcagcagctccagtaacctcaagacccacctgcgcctgcactccggggcccggcccttccagtgcagtgtctgccggagtcgcttcacccagcacatccacctgaagctgcaccatcggctgcatgccccacagccctgtggcctggtgcacacccagctgcccctggcctctctggcctgccttgcccaatggcaccagggggcactagatcttatggcggtggcatctgagaaacacatgggctatgacatagatgaggtcaaagtgtcctcgacatcccaggggaaagcaagagcagtgagcctgagcagtgccgggactcccctggtgatggggcaggaccagaacaattaaaaatgtttcttctgtcaaaaaaaaaaaaaaaa

SEQ ID NO: 31 - DNA, 1599 bp, Homo sapiens
ggggaggtagcacaaagggaggaccatggtcttctaactgaggaacctgagcagatgccttgacccagaccagagacccaagccaaggaaagcccatgatcaccagagtaatggggatatgaaggaagaatcagctgcacaattaggttgttgtcataggcccatggccctgggaggtacagggggctccctgtcccccagcctggacttccagctcttccgaggtgaccaggtcttctcagcctgcagaccacttccagacatggtggatgctcatggcccatcctgtgccagctggctgtgtcccttgcccctggcaccgggcaggtctgcactgctggcctgcctacaggacctggacctgaacctgtgcaccccacagccggcacccctgggcacagacctgcagggcctccaagaggacgccttgagcatgaagcacgagccaccagggctgcaggccagctccaccgatgacaagaaattcacagtcaagtacccacagaacaaggacaagctgggaaaacagccagaaagagctggcgagggggccccctgcccagccttctcctctcataacagctcttccccaccaccgctgcagaacagaaagagccccagccccttggctttctgcccctgtccccctgtcaactccatctccaaggagctcccatttctcctccacgccttctaccctggatacccacttctcctgcctccacccaacctgttcacctatggggccctaccttctgaccaatgtccccacctcctcatgctgccccaagacccctcctaccccaccatggctatgcctagcctgctgatgatggtcaatgagctggggcaccccagcgctcggtgggagaccctgcttccctacccaggggccttccaagcctctggccaagctctgccttcccaggcccgaaatccaggtgctggagctgccccaaccgactccccaggcctggagcgtggtggcatggcatctccagcaaagcgggtcccattgagttcccagacaggcaccgcagccttgccttacccgctgaaaaagaagaatggcaaaatcctgtacgagtgcaacatatgtggcaagagctttgggcagctctccaatctcaaggtccacctgcgtgtgcacagtggagagcgtccattccagtgtgccttgtgccagaagagcttcactcaacttgcccacctgcagaagcaccacctggtgcacactggggagcggccccacaagtgctcggtgtgccacaagcgcttcagcagctccagtaacctcaagacccacctgcgcctgcactccggggcccggcccttccagtgcagtgtctgccggagtcgcttcacccagctgcccctggcctctctggcctgccttgcccaatggcaccagggggcactagatcttatggcggtggcatctgagaaacacatgggctatgacatagatgaggtcaaagtgtcctcgacatcccaggggaaagcaagagcagtgagcctgagcagtgccgggactcccctggtgatggggcaggaccagaacaattaaaaatgtttcttctgtcaaaaaaaaaaaaaaaa

SEQ ID NO: 32 - DNA, 1645 bp, Homo sapiens
ggggaggtagcacaaagggaggaccatggtcttctaactgaggaacctgagcagatgccttgacccagaccagagacccaagccaaggaaagcccatgatcaccagagtaatggggatatgaaggaagaatcagctgcacaattaggttgttgtcataggcccatggccctgggaggtacagggggctccctgtcccccagcctggacttccagctcttccgaggtgaccaggtcttctcagcctgcagaccacttccagacatggtggatgctcatggcccatcctgtgccagctggctgtgtcccttgcccctggcaccgggcaggtctgcactgctggcctgcctacaggacctggacctgaacctgtgcaccccacagccggcacccctgggcacagacctgcagggcctccaagaggacgccttgagcatgaagcacgagccaccagggctgcaggccagctccaccgatgacaagaaattcacagtcaagtacccacagaacaaggacaagctgggaaaacagccagaaagagctggcgagggggccccctgcccagccttctcctctcataacagctcttccccaccaccgctgcagaacagaaagagccccagccccttggctttctgcccctgtccccctgtcaactccatctccaaggagctcccatttctcctccacgccttctaccctggatacccacttctcctgcctccacccaacctgttcacctatggggccctaccttctgaccaatgtccccacctcctcatgctgccccaagacccctcctaccccaccatggctatgcctagcctgctgatgatggtcaatgagctggggcaccccagcgctcggtgggagaccctgcttccctacccaggggccttccaagcctctggccaagctctgccttcccaggcccgaaatccaggtgctggagctgccccaaccgactccccaggcctggagcgtggtggcatggcatctccagcaaagcgggtcccattgagttcccagacaggcaccgcagccttgccttacccgctgaaaaagaagaatggcaaaatcctgtacgagtgcaacatatgtggcaagagctttgggcagctctccaatctcaagtggagagcgtccattccagtgtgccttgtgccagaagagcttcactcaacttgcccacctgcagaagcaccacctggtgcacactggggagcggccccacaagtgctcggtgtgccacaagcgcttcagcagctccagtaacctcaagacccacctgcgcctgcactccggggcccggcccttccagtgcagtgtctgccggagtcgcttcacccagcacatccacctgaagctgcaccatcggctgcatgccccacagccctgtggcctggtgcacacccagctgcccctggcctctctggcctgccttgcccaatggcaccagggggcactagatcttatggcggtggcatctgagaaacacatgggctatgacatagatgaggtcaaagtgtcctcgacatcccaggggaaagcaagagcagtgagcctgagcagtgccgggactcccctggtgatggggcaggaccagaacaattaaaaatgtttcttctgtcaaaaaaaaaaaaaaaa

SEQ ID NO: 33 - DNA, 1438 bp, Homo sapiens
ggggaggtagcacaaagggaggaccatggtcttctaactgaggaacctgagcagatgccttgacccagaccagagacccaagccaaggaaagcccatgatcaccagagtaatggggatatgaaggaagaatcagctgcacaattaggttgttgtcataggcccatggccctgggaggtacagggggctccctgtcccccagcctggacttccagctcttccgaggtgaccaggtcttctcagcctgcagaccacttccagacatggtggatgctcatggcccatcctgtgccagctggctgtgtcccttgcccctggcaccgggcaggtctgcactgctggcctgcctacaggacctggacctgaacctgtgcaccccacagccggcacccctgggcacagacctgcagggcctccaagaggacgccttgagcatgaagcacgagccaccagggctgcaggccagctccaccgatgacaagaaattcacagtcaagtacccacttctcctgcctccacccaacctgttcacctatggggccctaccttctgaccaatgtccccacctcctcatgctgccccaagacccctcctaccccaccatggctatgcctagcctgctgatgatggtcaatgagctggggcaccccagcgctcggtgggagaccctgcttccctacccaggggccttccaagcctctggccaagctctgccttcccaggcccgaaatccaggtgctggagctgccccaaccgactccccaggcctggagcgtggtggcatggcatctccagcaaagcgggtcccattgagttcccagacaggcaccgcagccttgccttacccgctgaaaaagaagaatggcaaaatcctgtacgagtgcaacatatgtggcaagagctttgggcagctctccaatctcaagtggagagcgtccattccagtgtgccttgtgccagaagagcttcactcaacttgcccacctgcagaagcaccacctggtgcacactggggagcggccccacaagtgctcggtgtgccacaagcgcttcagcagctccagtaacctcaagacccacctgcgcctgcactccggggcccggcccttccagtgcagtgtctgccggagtcgcttcacccagcacatccacctgaagctgcaccatcggctgcatgccccacagccctgtggcctggtgcacacccagctgcccctggcctctctggcctgccttgcccaatggcaccagggggcactagatcttatggcggtggcatctgagaaacacatgggctatgacatagatgaggtcaaagtgtcctcgacatcccaggggaaagcaagagcagtgagcctgagcagtgccgggactcccctggtgatggggcaggaccagaacaattaaaaatgtttcttctgtcaaaaaaaaaaaaaaaa

SEQ ID NO: 34 - DNA, 1725 bp, Homo sapiens; identical to SEQ ID NO: 29

SEQ ID NO: 35 - DNA, 1387 bp, Mus musculus
ttgctacaagatgaaagccctggatggtttaagagagtccctgtaccccagtctggacttccagctctatcaagatgaccaggtctgctcagctgacgcttctcagccacttgcagactcagtgggtgctcatgacttagcctggagcgagaggatgtgccccttaccactggcaccagctaaatctcccttgctggcctgccccgaaagcccggacctatgcttgtgtgccctgcagaagacacccctaggcagagccccacaggacctcggagaagacgcctcaaacatgagacaccagccgccaagcttgtacaaagcctccaccgacagtgagaaactcacaatcaaggactcactgaacagagaggagatgggaaatgagccagaaagaggcgcctacccacacctccctccccgtactagctccttcccagatgctgggctggacagaaagagccttagtcccttgaccttctggccctggcttcctcccactctcatctccaaggaaccccctatccacatctaccccatcttccctgggtacccacttctgcctctcccttacctattcacttatggggccctaccttctgcccaacatccatacctcttcatgctgcccccacactccacataccccaccgtggctgggcccagcttgcttatgacagccagtgggtctggacctcgtattccccaagagaagaccctgcttctccactcgggagctttccagagtgccggccacaccctacactcccaggttgagagtcggagctctagagatacccggaccccaggacaagctggtgtggcagctccaacaaggcgggctgtgccaggctcccgggcaggtgtcatagccctgccctaccctctgaagaaagagaatggtaaaatcttatacgagtgtaacgtgtgtggcaagaactttgggcaactctccaacctcaaggttcacctgcgagtacacagtggcgagcgcccgttccagtgtgccctgtgtcagaagcgcttcacccagctggcccacctgcaaaagcaccacctggtgcacaccggggagcggccccaccagtgccaggtgtgccacaagcgcttcagtagctccagcaacctcaagacccatcttcggctgcactcaggtgccaagccctcccagtgcggcctgtgtccaagttacctcacaccgaatgtctaccctaagctgcaccacaggctgcgggctcctcagctccgtggcctgacccacacccacctacctctcgcctctctcacctgccttgcccagtggcaccaaggagcgctagatcttgtggagaagaagatgggctggactgtggacaaggtgtcctcggaatccaagggaaagcaggggtag

SEQ ID NO: 36 - DNA, 1325 bp, Mus musculus
ttgctacaagatgaaagccctggatggtttaagagagtccctgtaccccagtctggacttccagctctatcaagatgaccaggtctgctcagctgacgcttctcagccacttgcagactcagtgggtgctcatgacttagcctggagcgagaggatgtgccccttaccactggcaccagctaaatctcccttgctggcctgccccgaaagcccggacctatgcttgtgtgccctgcagaagacacccctaggcagagccccacaggacctcggagaagacgcctcaaacatgagacaccagccgccaagcttgtacaaagcctccaccgacagtgagaaactcacaatcaaggactcactgaacagagaggagatgggaaatgagccagaaagaggcgcctacccacacctccctccccgtactagctccttcccagatgctgggctggacagaaagagccttagtcccttgaccttctggccctggcttcctcccactctcatctccaaggaaccccctatccacatctaccccatcttccctgggtacccacttctgcctctcccttacctattcacttatggggccctaccttctgcccaacatccatacctcttcatgctgcccccacactccacataccccaccgtggctgggcccagcttgcttatgacagccagtgggtctggacctcgtattccccaagagaagaccctgcttctccactcgggagctttccagagtgccggccacaccctacactcccaggttgagagtcggagctctagagatacccggaccccaggacaagctggtgtggcagctccaacaaggcgggctgtgccaggctcccgggcaggtgtcatagccctgccctaccctctgaagaaagagaatggttcacctgcgagtacacagtggcgagcgcccgttccagtgtgccctgtgtcagaagcgcttcacccagctggcccacctgcaaaagcaccacctggtgcacaccggggagcggccccaccagtgccaggtgtgccacaagcgcttcagtagctccagcaacctcaagacccatcttcggctgcactcaggtgccaagccctcccagtgcggcctgtgtccaagttacctcacaccgaatgtctaccctaagctgcaccacaggctgcgggctcctcagctccgtggcctgacccacacccacctacctctcgcctctctcacctgccttgcccagtggcaccaaggagcgctagatcttgtggagaagaagatgggctggactgtggacaaggtgtcctcggaatccaagggaaagcaggggtag

SEQ ID NO: 37 - DNA, 11141 bp, Homo sapiens
ggggaggtagcacaaagggaggaccatggtcttctaactgaggaacctgagcagatgccttgacccagaccagagacccaagccaaggaaagcccatgatcaccagagtaggttgggaagtgcggatagaggggaagtagtttttatttttttctaacagagaagttgtgcaaggtgttctgagagttgggagcagagtgtgatgccaggagagggcagagctggcagatggaagccaaaggtgtgcgggtcaccccttctgggtggggacatctgatcgacctagtccatgtatgcctgcatgaagtgctggtgagggaggttggaattaagtatttggccatttcggcttttataatgggaggtaccttttgcctccctcaagactcatgtaggggaaaagtttcagaggctgggcgtccaaaaaaacctgacagatgtctacatggaaaaccaaaatgagtagacagaggaccctgtccttgagatgctcacaaacatgttgggatcagttatgtggaggatgagtcacagccctaggggctgagctggcagagaaaccccaggaacatgccccagcccatggtgcacagcctgagatgtccctgtatattctgggacaccagggtggcgtggctggggtgcagattgatggctggttggggcttttgcaccctgggccagactcacgcacaccagaatttgtgtaaggccgcaggggtgatgcctcttctcatgctcacaagaataatgacattgctcaggctaaaaagccactttctggcctatgaggtctccctttttgtatctagacatgtcttttccttatttattaactttttttaagacagggtctcgctctgtcacccaggctggagtgcagtggcatgatctcagctcactgcagtctcgacctccctggctcaagtgatcctcccacctcagcctcctgagtagctggaacaacaggcatgcgctaccatgcctggctaatttttgtattttttgtagagatggtgtttcaccatgttgctcatgctggtcttgaactcctgggctcaagcagttggcccacctcagcctcccaaagtgctgggattacagatgtgagccaccacacctggcctagtcatgccttacacccattttgctatgtctattttttttttttttaaagagacagggtctttctctgtcacccaggctagagtgcagtggcaatatcctagttcactgcagccttatattcctgggctcaagtgatcctcctgcctcagcctcctgagtagctgggaccagaggtgcaagtcaccatgcccagctaagttttgtattttttgtagagatgaggtcttgctttgttgcccaggctgatctcaaactcctgggctcaagagatgggtctgccttggtctcctgaagtgctgggattacagatgtgagccacttcacctggccatgtgcttgaattatcttttgcctcttggggcctctagattagaggatagaactttccagggtcaggagatctgagatgggacttggaggaggaaaacgtgtttgctgtatctctgtgagatcagctgccttctctgtcctctgtctcattccatatccttctatgttgcctgttcttgtctctgcctgttcctgtccctacctgtcaccatcactgtctccctgtcatcatcaatgcctatcttcatctcagcttggctcccctccacagaaggctcccctgttcagcctttgagccctactctgtcctcccgtccagccatctgggtactttgagaacagaaataacagcccgcccccctcacacacacatcctgaaattccagtagccccagttcactgcaatccacatcagcaggttgtcctccagagttgccacaggtaggcaaagctgagtggatagtgcccccttgtcagaccttgcagggcccccaagcctgcctgcccccacttcctatctgtggcaacttcaaatgtaacaccttcctgccatctctcactcccactggggcctcagccacaggcagctgtggcctggctgcccacctccttccttcccagccaggagcactgctgcagcaccgagtggagagccccacagccagcagcccttcgggggtccatggtatttgctttgagacctgaagcagcgttgggaggagtgaggaaagagggaaaggagaaggggttttagagggctgagacactggagtgagggtcttagggtctgaggggcatttgggattcaaagagtggtccctgaggtgacggggaaggggtcttggggcatttgctgggctgaagtgttccctgtgctgtgtggcctcaggtggaagatggcagagaagcctggtgctggatgagcagacagctgggcactcgcgtctctctgtcaggccagtcaggggcatccgggggcttggtccactcattcaggctttattcctggtgagagcgctgagctgggcagcctgggtgacacgacactgaggtgtcctcagctgtccagaagagctatctgggtgagctaggatgcaggattccccttggtggggggcctctggttggtagagtcagggacccctctcagcacagaagggggtggcttctttgtagccctgaggaatctggaatgttccctcagatctctgcacttacttggaagtaagctccgtgagggccttcctctcctctcaggagagctctgctcttcacttctgattggctaatctccattgattgattgattgattgattgattgagacagggtcttgctctatcctcgcccaggctagagtgtagtggtgtgatctcagctcactgcagccacaaccttccaggctcaagcaatcttcccacctcagcctcccaagtagctgggaccacaggcacaggctactacacccagctaatttatttatttatttttgaaccgagtctcactctgttgctcagactggagtgcagtgacatgatctcggctactgcaacctccgcctcttgggttcaagcgattctcttgcctcagcctccctagtagctgggactacaggtgtgagccaccacactcagctaattttttttttttggagacaaagtcttgctctgtcacccaggctagaatgcagtggtgctatcttggctcactgcaacctctgcctcccgggttcaagcgattctcctgcctcagcctcctgagtagctgggattacaggcgtgcgccaccacacccggctaatttatgtagttttggtagagacgaggtttcaccatgttcgtcaggctggtcttgaactcccaacctcaggtgatccgcccgcttcagcctcccaaagtgctgggattacaggcatgagccaccgcacctggtctaattttaatttttggatttttagtagagatgggggtttcaccatgttagccaggctggtctcgaactcctgacctcaggtgatccatctgcctgggtctcccttttattttttatttttttatttattttattttatttttagaaggagtctcattctgttgcccaggcttgaatgcagtggcacgatcttggctcactgcaacctccacctcccaggttcaagtgattcttgtgccttggcctcctgagtagctgggattataggcatgcaccaccgtgactggctaatttttgtatttttagtagagacagggtttcaccatgttgccgaggttagtcttgaactcctgacatcaggtcatccaaccgccttggactcccaaagtgctgggattacaggcctgagccaccccgcccaccatattttttattttgtagagatgggggtctcactatgttgcccaggctggtctagaactcctggcctcaaaagatccttccttctcagcctcccaaagtgctgggattacaggcttgagccacggcacctggccaatctctatttaatcttcccacacccaccaggagagcatcactggcttctaaatgtcaacattgagagaatccttacgcatcaactagaccaaccttcccattttgcggatagggaaactgagtccaggaagggacatgaataacttgtggtcatgcagcatgttaatgggagagccagtcctagaactcagactcctatttggaggaccttcacagcttacctctaactcacctgtttcccagcaggtaatggggatatgaaggaagaatcagctgcacaattaggttgttgtcataggcccatggccctgggaggtacagggggctccctgtcccccagcctggacttccagctcttccgaggtgaccaggtaaggtagaaccccagccttacctttgtgggtagtctgcttatagtaggaggtgacattaggaggaaaaaggggaacgtctgaagggacctagatctccttcccagtctgagcaccccctatcccaggcacatcatagggatgtttctttgaggagccagacaccctctccccatgcctcaggggcccagactctcctgtggggaataagaagcggaagcctcgtgcagtaggcttggctgctatgaccatggccttgggccttagtgagcaggggctcagggccatcttgtggagtttatagtctttgatggggattttctttgtgctcaacttggtgccattcaaattacagtgatggtctgagatacactggggaccagggtggtcagaggtacatgggttcggggcagggagggagaacagggctagagagggccctggggacaggaggagggcaatcttgtggtgggtgggaaaatcctgggggaaagagatctttggatgagggaagagatgccatttgagtgacagatatggagtgtggtggctgttttagcttattctcccctttggaaaacaaaataggggtaggggccatctaagggacccatggagtcccagtcaccagcccccaagccccttgcccctgccctccaggtcttctcagcctgcagaccacttccagacatggtggatgctcatggcccatcctgtgccagctggctgtgtcccttgcccctggcaccgggcaggtctgcactgctggcctgcctacaggacctggacctgaacctgtgcaccccacagccggcacccctgggcacagacctgcagggcctccaagaggacgccttgagcatgagtaagcccagggcaccgggctgcacctgcgcctggcagaggctggagggtggggggcactgctaggtttggggatgggtggtggagaggacgcagtgtcactgatgaagacagagcttaggcacactttacttccacattttggccttggagttctggcctgagggtgcagggggctttcgagtggtgatacctaagccagagaagctgggcactggcttctcaggcatgtgtgtgggagctgatgtaggtatagccaaggtcatgggggttggggatggtgctttgtctgcttagagaggtgttttttgtttgtttgtttgtttttgagatggagtctcgttctgttgcccaggctggagtgcaatggcatgatctccagtcaccgcaacctctgcctcccaggttcaagcgattctcctgcctcagcctctcggagtagctgggattacaggtgtgcgccaccacccccggctaatttttgtatttttagtaaagacagggttttgccatgttggccaggctggtctcgaactcctgacctcaggtgatccacctgcctcggtgacccaaagtgctggaattatagatgtgagccactgtgcccagccctagagggatcttataaaagtactggaggccgggcacggtggctcatgcctgtaatcccagcactttgggaggctgaggcgggcacatcacgaggtcaggagatcgagaccatcctggctaacacggtgaaaccccatctctactaaaaatacaaaaaattagctggggggtggtggcaggtgcctgtagtcccagcgactcaggaggctgaggcaggagaatggcatgaacctgggaggcggagcttgcagtgagccgagatggcaccactgcactcagcctgggcgactgagcgagactccatctcaaaaaaaaagcactgggctgggagtcagggaattggggttggagtctcggttctgccctttctatttctatggacttggacacaacaccaaggtctggtttcttcatccataaaagtgtgatctcatctctgcctgggttctaggtttgttgtaacacaccatttccttatgtgacgatgaccccctgaaggcaaggactctcttgctgtgtcctttatttctaggaccccagcacagagcacagagtgaaacggctcagtgagattgattgagtaaataaataaggatccaaccagaagacaacttgctttttttttttttttttttttttgagataggctggagtgcagggacacaatcatggctcactgcagcctccaactcctgggctcaagcaatcctctctcctcagcctcctgggtagctgggactactacagacacatgccacaaacacccagctaattttctttctttctttctttcttttttttgaaacagagtttcgcttttgttgcccaggctggagtgcaatggcatgatctcggctcaccgcaatctctgcctcccgggttcaagtgaatcttctgcctcagcctcccaagtagctgggattacaggcatgtgccaccatgcccagctaattttgcctttttagtagagacggggtttctccatgttggtcaggctggtcttgaactcccaacctcaggtgatcttcccgcctcggcctcccaaagtgctgggattacaggcgtgagccaccgtgcccagccttaattttcttattgttttgtagagatgggattttgctttgttgcccaggctggtctcaaattcctaggcttaagcaatcctcctgcctcagcctcccaaagcactgggattataggcatgagccactgcacctggctgacagtttattttttaacaggttttttattttagtactctttatctagaatagcactgtttggtagaactccctgcaataatgggaacattctatacctgtgctgtccaatactatacctgctgagcactgtaaatgtgactggtgcaactgagaacttcattttttattagaatcattgtaattaatgtaaatctagtcatgcatccagtggctgccatttcggacatagcagatctagcctcacccttatgtgatcttatctgtgcccatggcacctggatttatgtctccagttcagacctctcctttaaactccaacccagtttatccatctgcccacttggcttctcaacctgggtgtctaatagggaccttgaactgctttctccccctgaaattgttcagcactacagaaatgctagcaggctcgcaggctggaggtctttctcagccccgagcagggcaggtttgatggcggacagaacttcaccccagactgtggggatctgaccgccttccttttcctgcttagagcacgagccaccagggctgcaggccagctccaccgatgacaagaaattcacagtcaagtacccacagaacaaggacaagctgggaaaacagccagaaagagctggcgagggggccccctgcccagccttctcctctcataacagctcttccccaccaccgctgcagaacagaaagagccccagccccttggctttctgcccctgtccccctgtcaactccatctccaaggagctcccatttctcctccacgccttctaccctggatacccacttctcctgcctccaccccacctgttcacctatggggccctaccttctgaccaatgtccccacctcctcatgctgccccaagacccctcctaccccaccatggctatgcctagcctgctgatgatggtcaatgagctggggcaccccagcgctcggtgggagaccctgcttccctacccaggggccttccaagcctctggccaagctctgccttcccaggcccgaaatccaggtgctggagctgccccaaccgactccccaggcctggagcgtggtggcatggcatctccagcaaagcgggtcccattgagttcccagacaggcaccgcagccttgccttacccgctgaaaaagaagaatggcaaaatcctgtacgagtgcaacatatgtggcaagagctttgggcagctctccaatctcaaggtatctgcctcctgggctctgcttctcccctcaacatccttcagggccccccatgcacccgagtctgcttctagaagcaaggacctcaaagctagaaggcactcctgggcaacctctagggccaagtgaccttggatggttcatttgggtggagatatcggataagacggccaccaaagtcctcgccaccctagaggactgcctatgagatcgtctcatttaggttaaaatggggagactgaggcttttaatgggcagcgtttgcctaagattaccccgatttaacggtagtgttaggtttagtctctcaacatttgctctgggcaaagaaagcccttacctggacaaccatcctttctggactccaagttaagcttcttatttatttttttgggcagtcagatgagggaatgggtagattttggtgagtctagaccacagtccgatgaccaacctttttcaagtgggatcccacaaatctgcgcgaccgccgatgcgattggcctcaccatccgcatcgcccagcagggggcgcccagggagcgcatctcagtgcgttagcaaagggcggaaactgtgcgctctctggctagttctgaagttgaaggcacattgtggttatggggagaagccctgctggatcctgcagggcaggagttcttaaactttcatgagcctcagaccacctggagagctagtaaacatgcagattcctagagtttttgtgatcctcccacctcagcctccagagtagctcccagccccagagttatcttttttttttttttttttttccatatggagtctcattctgtcgcccacgctggtctcaaactcctgggcttaagggattctcccacttcatcctccccggtagctgggacgaagggtaaacaccaccactcctcgttaactttcaaattttttgtgcatacggggtcccaatgtgtttctcaggctggtattgaactcctgggctccattgatcctccctcctcggcctcccaaaatgctgggattacagccacgagagtgtgcccagccctcccagagtttttgattcagcaggtctggggccggacttgagaacctgcatttctaacatgctcccaaagtgtgataccagcactgctggtctgtgaaccacattttgaatagcagtgttccacaatatacctggggtacctcttacccctggccttctgaccactgccatctcccctacagggcagcagaaagagtagagtatcctggggatgatgggccctgagggagatgacggggagggagagagaagtgggcagagatttggagaggacctcaggccctgctaggggtgctgggcagttggcagcatttatcccgcatctgactcaggtccacctgcgtgtgcacagtggagagcgtccattccagtgtgccttgtgccagaagagcttcactcaacttgcccacctgcagaagcaccacctggtgcacactggggagcggccccacaagtgctcggtgagatccttctccccctactatgtactcctataggctggagaggagttttaggcccttggagggctggcacctgcagcctccattccccagtcctcctgagcaagtggagatgggaagggggagtgaaaattgaaaatgactcctctcagcctggaatgggtaatactgaaggtcactccaaaggcctgcaggagggagagaacaatgaagttggaggtcatggagagtgtatgtatacccattatctacctccctgagttattggaggagttaatgctatagtgtacccagagtatccaagtaggaaaactattagcttgatgtcagctccctccttctgtgagcctggccacatccctcgggaaatcagaagggcaggggttctttcccattttacaggggcagaaatgaaagcagtgccatgattatgagtccgggctgcatagtcgggccacctgtgttcaaatcctagctctgccttcaacttgctgtgtgacctttggctagtggcttcactcctctgagactctgctttcttactataatataggacttgtactaatacttagtcatgcttccctagggtcctggggaagactagatgaaaccaactgcaggcatgtggcagccccctgtaaatgttcattacaatctactgaagctgcagcttcatacttaagagttctatgtgattatctcattcagtccaccatatctatgaagctaggtattaataaccaatgttaccaatgaggagaggaaggctcagagaggagaagttacttttacaaggtcacacatggagtaaaggacactaacccaagttggcctggttctaatgtgctagataccgtgggttccaggaagaaatcactggaaaagcttccaggcctggagggaaagagaggtgggtcatgggtatttccaagatgaggagagggagtagaatgcctgagccagcccagaggacaaagctgatgccagtgctcgttcccgtcaggtgtgccacaagcgcttcagcagctccagtaacctcaagacccacctgcgcctgcactccggggcccggcccttccagtgcagtgtctgccggagtcgcttcacccagcacatccacctgaagctgcaccatcggctgcatgccccacagccctgtggcctggtgcacacccagctgcccctggcctctctggcctgccttgcccaatggcaccagggggcactagatcttatggcggtggcatctgagaaacacatgggctatgacatagatgaggtcaaagtgtcctcgacatcccaggggaaagcaagagcagtgagcctgagcagtgccgggactcccctggtgatggggcaggaccagaacaattaaaaatgtttcttctgtc

(19) United States
(12) Patent Application Publication
(10) Pub. No.: US 2011/0159020 A1
(43) Pub. Date: Jun. 30, 2011

(54) Transcription Factor for Killer Cell Activation, Differentiation and Uses Thereof

(76) Inventors: Rene Antonius Wilhelmus van Lier, Uithoorn (NL); Amber van Stijn, Amsterdam (NL)

(21) Appl. No.: 12/518,061

(22) PCT Filed: Dec. 7, 2007
(86) PCT No.: PCT/NL07/50638

§ 371 Date: Dec. 14, 2009

(30) Foreign Application Priority Data
Dec. 7, 2006 (EP) EP 06077192.0

(51) Int. Cl. A61K 31/7088; C12N 15/85; C07H 21/04; C12N 15/63; C12Q 1/02; A61P 37/04

(52) U.S. Cl. 424/185.1; 435/455; 536/23.5; 435/320.1; 536/23.4; 435/29

(57) ABSTRACT

The present invention provides methods for altering activation and/or differentiation of Killer cells comprising altering the expression of a Homologue Of Blimp-1 In T-cells (Hobit) species in said Killer-cells. The invention further provides methods and means for increasing or decreasing expression of a Hobit species in Killer cells, and provides isolated and/or recombinant nucleic acid and uses of said isolated and/or recombinant nucleic acid.

The invention relates to the field of immunology. The invention in particular relates to means and methods for modulating an immune response in an individual.

The hemopoietic system is a complex system containing a large variety of different cell types. These cells include the very abundant and differentiated red blood cells, B- and T-cells, monocytes and macrophages. These differentiated cells often have a limited time span and need to be replenished continuously. This is achieved through a cascade of increasingly more primitive cells that have self-renewal capacity and differentiation capacity. In general it can be said that the more primitive the precursor cell the more self-renewal capacity the cell will have and the more different cell types the precursor can generate. The top of the cascade is formed by the so-called pluripotent stem cell that has extensive self-renewal capacity and the capacity to generate every differentiated hemopoietic cell of the adult system. This stem cell can, through differentiation into a series of more committed progenitor cells, provide the hemopoietic system with the various types of differentiated cells. Stem cells and progenitor cells are preferred examples of precursor cells as used herein.

Many different markers have been identified with which the various types of hemopoietic cells can be distinguished from one another. Also many of the growth factors and other external signals that direct the process of differentiation have been identified. However, although much is known about the various differentiated cells and their precursors; surprisingly little is known about the nuclear factors that are the mediators of the differentiation process.

An exception to this rule is perhaps red cell differentiation. Here several different transcription factors have been identified that at least play a role in the expression of the globin gene during development and during differentiation of the committed red blood cell progenitors. In the B-cell lineage, a transcription factor called Blimp-1 has been identified. This factor is involved in the maturation of B-cells into plasma cells. In the present invention a Homologue Of Blimp-1 In T-cells (Hobit) has been identified. Analysis of mRNA in Hobit expressing cells revealed the presence of a number differently spliced variants (further referred to as Hobit species). The amino acid sequence of the different splice variants is given in FIG. 8 or 9.

Expression of Hobit species is induced in response to activation and/or differentiation of Killer-cells and/or when an infection needs to be combated. Vise versa, Hobit expression in resting Killer cells and/or naïve T-cells activates and/or differentiates these cells.

All Killer cells ultimately derive from haematopoietic stem cells in the bone marrow. Hematopoietic progenitors derived from haematopoietic stem cells populate the thymus and expand by cell division to generate a large population of immature “thymocytes”. The earliest thymocytes express neither CD4 nor CD8, and are therefore classed as double negative (CD4⁻CD8⁻) cells. As they progress through their development they become double positive thymocytes (CD4⁺CD8⁺), and finally mature to single positive (CD4⁺CD8⁻ or CD4⁻CD8⁺) thymocytes that are then released from the thymus to peripheral tissues.

About 98% of thymocytes die during the development processes in the thymus by failing either positive selection or negative selection, while the other 2% survive and leave the thymus to become mature immunocompetent T cells. In bone marrow transplantations it has been observed that stem cells from the graft repopulate also the T-cell lineage. As bone marrow transplantation is often performed in adults that no longer have a fully developed thymus, it is thought that replenishment of the T-cell lineage and education of the T-cell such that they do not recognize the “self”-antigens of the host continues throughout the life of an adult, albeit at a low level in normal situations. Macrophages are part of the myeloid lineage and also originate from the pluripotent hemopoietic stem cell. They do not pass through the thymus. NK cells also originate from the pluripotent hemopoietic stem cell and are thought to derive from a lymphoid progenitor they share with B-cells and T-cells.

T-cell activation varies slightly between the different types of T-cells; however, the following two-signal model is generally true for most: The interaction between TCR molecules and specific MHC/antigen complexes on antigen presenting cells (APCs) delivers signal 1 into the T cell. Co-stimulatory interactions between CD28 molecules on the T cell and B7 molecules on the APC deliver signal 2, activating the T cell. Without co-stimulation a T cell will become functionally inert (anergic). T cell activation can be followed by measuring proliferation, usually by incorporation of radioactive thymidine, or secretion of IL-2, an event which is dependent on co-stimulation.

A naive T cell is considered mature, but is distinguished from activated T cells or memory T cells, as it is thought to not have yet encountered cognate antigen in the periphery. Naive T cells are commonly characterized by the surface co-expression of the surface markers L-selectin (CD62L) and CD45RA; the absence of the activation markers CD38 and CD69; and the absence of memory markers, such as the CD450 isoform. In the naive state, T cells are thought to be quiescent and non-dividing, requiring the common-gamma chain cytokines IL7 and IL15 for homeostatic survival. Naive T cells are able to respond to novel pathogens that the immune system has not yet encountered. Recognition by a naive T cell clone of its cognate antigen results in the initiation of an acquired immune response. In the ensuing response, the T cell acquires an activated phenotype (IL7R−CD38+CD45R0+), and may further differentiate into a memory T cell (CD38−CD45RO+).

Memory T cells are a specific type of infection-fighting T cell (also known as a T lymphocyte) that can recognize antigens such as bacterial, viral and tumour specific antigens, that were encountered during a prior infection or vaccination. At a second encounter with the antigen, memory T cells can reproduce to mount a faster and stronger immune response than the first time the immune system responded to the antigen. There are several distinct populations of memory T cells, based on phenotypic and functional properties. Memory T cells can be recognized by the differential expression of certain molecules. Central memory T_{CM} cells express L-selectin and the chemokine receptor CCR7, they secrete IL-2, but little IFN_{Y} or IL-4. Effector memory TEM cells, however, do not express L-selectin or CCR7 but produce effector cytokines like IFN_{Y} and IL-4. Antigen-specific memory T cells against viruses or other microbial molecules can be found in both T_{CM} and T_{EM} subsets. Although most information is currently based on observations in the Cytotoxic T cells (CD8-positive) subset, similar populations appear to exist for both the Helper T cells (CD4-positive) and the cytotoxic T cells.

Within the human cytotoxic T cell population, three distinct sub-populations have now been described: central memory (T_{CM}), effector memory (T_{EM}), and effector memory RA (T_{EMRA}) T cells, giving two highly related effector memory sub-types. The T_{CM} cells are thought to represent memory stem cells. T_{EM} and T_{EMR}A cells strongly express genes for molecules essential to the cytotoxic function of CD8 T cells, whereas T_{CM} display a capacity for self-renewal due to high levels of phosphorylation of the transcription factor known as STAT5.

Natural killer (NK) cells are a form of cytotoxic lymphocyte. Cytotoxic lymphocytes constitute a major component of the innate immune system. Within the context of the innate immune system, NK-cells act to lyse cells in a way that does not require the MHC-presentation of a specific “disease” associated antigen. NK cells were discovered due to their non-MHC-restricted killer activity that disturbed early attempts to generate tumour-specific, MHC-restricted cytotoxic T lymphocytes (CTLs) from mice shortly after the discovery of the phenomenon of MHC restriction. NK cells are large granular lymphocytes that do not express T-cell antigen receptors (TCR) or Pan T marker CD3 or surface immunoglobulins (Ig) B cell receptor but which usually express the surface markers CD16 (Fc_{Y}RIII) and CD56 in humans, and NK1.1/NK1.2 in certain strains of mice.

With the discovery of activating receptors almost two decades after the discovery of the inhibitory receptors these cells continue to be called by the same name, though “natural” does not mean the same any more. However the term “natural killer” continues to be justified by:

NK cells are cytotoxic; small granules in their cytoplasm contain special proteins such as perforin and proteases known as granzymes. Upon release in close proximity to a cell slated for killing, perforin forms pores in the cell membrane of the target cell through which the granzymes and associated molecules can enter, inducing apoptosis. The distinction between apoptosis and cell lysis is important in immunology—lysing a virus-infected cell would only release the virions, whereas apoptosis leads to destruction of the virus inside.

NK cells are activated in response to type 1 interferons or macrophage-derived cytokines. They serve to contain among others viral infections while the adaptive immune response is generating antigen-specific cytotoxic T cells that can clear the infection. Patients deficient in NK cells prove to be highly susceptible to early phases of herpes virus infection. NK cells are currently also investigated as killer cells for the destruction of tumours.

In order for NK cells to defend the body against viruses and other pathogens, they require mechanisms which enable the determination of whether a cell is infected or not. The exact mechanisms remain the subject of current investigation, but recognition of an “altered self” state is thought to be involved. This “altered self” state also seems to be present on at least some types of tumour cells.

To control the cytotoxic activity, NK cells possess two types of surface receptors: “activating receptors” and “inhibitory receptors”. Most of these receptors are not unique to NK cells and can be present in other T cell subsets as well.

These inhibitory receptors recognize MHC class I alleles, which could explain why NK cells kill cells possessing low levels of MHC class I molecules. NK cell receptor types (with inhibitory as well as some activating members) are differentiated by structure:

CD94: NKG2 (heterodimers)—a C-type lectin family receptor, conserved in both rodents and primates and identifies non-classical (also non-polymorphic) MHC I molecules like HLA E. Though indirect, this is a way to survey the levels of classical (polymorphic) HLA molecules, however, because expression of HLA-E at the cell surface is dependent upon the presence of classical MHC class I leader peptides.

Ly49 (homodimers)—a relatively ancient, C-type lectin family receptor; are of multigenic presence in mice, while humans have only one pseudogenic Ly49; the receptor for classical (polymorphic) MHC I molecules.

KIR (Killer cell Immunoglobulin-like Receptors)—belong to a multigene family of more recently-evolved Ig-like extracellular domain receptors; are present in non-rodent primates; and are the main receptors for both classical MHC I (HLA A, HLA B, HLA C) and also non-classical HLA G in primates. Some KIRs are specific for certain HLA subtypes.

ILT or LIR (leucocyte inhibitory receptors)—are recently-discovered members of the Ig receptor family. Activation of NK cells is preferably determined by measuring Nkp44 and/or 46.

Macrophages differentiate from monocytes. When a monocyte enters the attacked tissue through the endothelium of a blood vessel (a process known as the leukocyte adhesion cascade), it undergoes a series of changes and becomes a macrophage. Macrophages are attracted to a damaged site by chemical substances through chemotaxis, triggered by a range of different stimuli including damaged cells, pathogens, histamine released by mast cells and basophils, and cytokines released by macrophages already at the site. The life span of a macrophage ranges from months to years. Macrophages are unable to divide and must mature from monocytes produced in the bone marrow. A monocyte is therefore considered to be one of the precursors of a macrophage.

The present invention provides a method for altering activation and/or differentiation of Killer cells comprising altering the expression of a Hobit species in said Killer-cells. In a preferred embodiment of the invention said activation and/or differentiation is elevated through increasing expression of said Hobit species in said Killer-cell. When Killer-cells of a collection of Killer-cells are provided with a Hobit species it allows activation of a resting Killer cell and/or naïve T-cell as measured by the expression of at least one activation marker for the specific Killer cell and/or T-cell. Killer-cells that are not fully activated become more strongly activated as measured by the increased expression of said activation marker. Fully activated Killer-cells proceed to a more differentiated effector phenotype as evidenced for instance in T-cells by the strong increase in IFNg secretion. A collection of Killer-cells with a certain overall activation level will become more activated as a result of the provision of said Hobit species. This process can be measured by an increase in the overall expression level of said activation marker. Differentiation of Killer-cells often, but not necessarily always, follows the activation state of the Killer-cells and can be measured using the same activation marker.

In another preferred embodiment said activation and/or differentiation is reduced through decreasing expression of said Hobit species in said Killer-cell. Reduction of activation and/or differentiation is achieved by lowering or decreasing the expression of said Hobit species in an activated and/or differentiated Killer-cell that expresses said Hobit species.

A lowering of the expression can be achieved in a number of different ways. Preferably, but not necessarily, said cell is provided with a nucleic acid that contains an anti-sense sequence for RNA encoding said Hobit species. There are several types of anti-sense strategies that can be used. Non-limiting examples are the provision of a cell with a siRNA, a miRNA, a hairpin RNA or DNA molecule, the use of oligonucleotides that can induce exon-skipping and other strategies. Many of these strategies use analogues of nucleic acid to increase delivery, stability or other factors to improve the efficiency of the reduction of level and/or translation of the target RNA molecule. Such modifications and/or analogues of nucleic acid are in the present invention functional equivalents of a nucleic acid containing an anti-sense sequence. Non-limiting examples of such functional equivalents are LNA, PNA, 2-O-methyl modified oligonucleotides, morpholino's and others. Functional equivalents are for instance also nucleic acids that contain one or more nucleotide analogues. Non-limiting examples of such nucleotide analogues are those that have been developed and that will be developed because of their effect on viral replication. Others such as inosine are developed for a different purpose. A general characteristic of a nucleotide analogue of the present invention is that it can be incorporated into a nucleic acid molecule be it in a synthetic process or in a natural process of nucleic acid synthesis, or in a combination thereof. A reduction of activation and/or differentiation of a Killer-cell is preferably measured by a lowering or reduction in the expression of said activation marker on said cell. A reduction of activation and/or differentiation of a collection of Killer-cells containing activated Killer-cells is preferably measured by measuring a lowering or reduction in the overall expression of said activation marker in said collection of Killer-cells.

Killer-cells can, as mentioned herein above, be replenished from the bone marrow in pre-pubertal individuals and also, albeit to a lesser extend, in post-pubertal individuals. In the context of the present invention it is possible to affect the activation and/or differentiation potential of Killer-cells formed in this process by providing a Killer-cell precursor of said Killer-cell with a nucleic acid for altering the expression said Hobit species in a progeny Killer-cell of said precursor. When provided with a nucleic acid or functional equivalent thereof comprising an anti-sense sequence of said Hobit species it is possible to reduce the potential for activation and/or differentiation of the resulting progeny Killer-cell. When the precursor of said Killer-cell is provided with a nucleic acid for enhancing and/or elevating expression of said Hobit species in the resulting Killer-cell, said Killer-cell becomes activated as a result of the elevated enhanced and/or elevated expression thereof.

In a preferred embodiment of the invention said nucleic acid is provided to a naïve T-cell, an NK cell or an antigen specific T cell. An antigen specific T-cell is preferably specific for a tumour and/or a virus. An antigen specific T-cell provided with a nucleic acid encoding a Hobit species of the invention will become activated and/or more differentiated when provided with said nucleic acid for enhancing and/or elevating expression of said Hobit species in said antigen specific T-cell. Similarly, a collection of antigen specific T-cells will become more activated and/or more differentiated. These cells are preferably used for adoptive therapy. A naïve T-cell provided with a nucleic acid encoding a Hobit species of the invention will become activated and/or more differentiated when provided with said nucleic acid for enhancing and/or elevating expression of said Hobit species in said naïve T-cell. Similarly, a collection of naïve T-cells will become more activated and/or more differentiated. A naïve T-cell provided with a nucleic acid and/or functional equivalent thereof, for lowering and/or reducing expression of said Hobit species is less inclined to become activated and/or differentiated when induced. Similarly, a collection of naïve T-cells provided with said a nucleic acid and/or functional equivalent thereof, for lowering and/or reducing expression of said Hobit species is less inclined to become activated and/or differentiated when induced. The activation state of said naïve T-cell and/or said collection of naïve T-cells, prior to or following the provision of said nucleic acid can be measured as described herein above.

The present invention is preferably applied in humans and/or human cells in vitro. However, other animals a such as but not limited to other mammals and even birds have a corresponding homologue of human Hobit. The present invention can therefore also be applied to (cells of) said other species. In a preferred embodiment said Killer-cell or precursor thereof is a human Killer-cell or human precursor thereof.

The mentioned cells are preferably in vitro provided with a means for altering expression of said Hobit species. Preferably said expression is increased in cells in vitro. A preferred application of the present invention is the improvement of the immune capability of a graft. Grafting of autologous, matched and mismatched cells has become a standard practise in the clinic for a variety of different diseases. Many of the grafts that are transplanted contain immune cells. These cells are often transplanted for the purpose of fortifying the immune system of the recipient. In many cases this is done to combat tumours in the recipient, Many of these transplantations show some effect of the transplanted immune cells on the tumour cells. In some cases the effect is indeed very impressive. However, often also the effect is transient and insufficient to remove a significant number of the tumour cells for the recipient. The transplanted immune cells are apparently often not sufficiently activated/differentiated to combat the tumour efficiently. In the art various strategies are pursued to increase the tumour killing potential of the grafted immune cells. In the present invention it was found that the activation and/or differentiation of immune cells in a graft is elevated and/or enhanced upon increasing expression of a Hobit species in immune cells and/or precursors thereof of said graft.

In one embodiment a method of the invention is used for enhancing a Killer-cell response of said Killer-cells or progeny thereof. In a preferred embodiment a method of the invention is used for enhancing an antiviral response and/or an anti-tumour response of said Killer-cells, preferably antigen-specific T-cells and/or NK-cells. A method of the invention is preferably used for enhancing an antigen specific T-cell response.

A preferred marker for measuring elevation of activation and/or differentiation comprises IFNg expression of the cells that were treated by a method of the invention.

Increasing expression of said Hobit species in a Killer-cell or precursor thereof results in enhanced and/or elevated activation and/or differentiation of said Killer-cell or the progeny of said Killer-cell and/or Killer-cell progeny of said precursor. The Killer-cell or the progeny of said precursor cell preferably a naive T-cell, a memory T-cell, an NK cell, a macrophage/monocyte, an effector T-cell, an antigen-specific T-cell, preferably specific for an antigen of a tumour or a virus infected cell, and/or a CD28-CD27-CD4 positive T-cell. The cells that have become more activated and/or more differentiated are preferably memory T-cells, NK cells, effector T-cells, antigen specific T-cells and/or CD28-CD27-CD4 positive T-cells.

In another aspect the invention provides an isolated and/or recombinant nucleic acid comprising a sequence as identified in FIGS. 8, 9 and/or 10, or a functional part, derivative and/or analogue thereof. In a preferred embodiment said nucleic acid encodes a human Hobit-L, Hobit-M, Hobit S and/or Hobit XS or a mammalian homologue thereof. A functional part is at least a part comprising the region containing the zinc fingers of a Hobit species. A derivative of a Hobit species is a Hobit species as depicted in FIGS. 8, 9 and/or 10, having between 1 and 3 amino acid substitutions. When these amino acid substitutions are in a zinc finger it is preferred that the amino acid substitution is done with an amino acid at the corresponding position in a zinc finger of another zinc finger protein. An analogue of a Hobit species and herein preferably a human Hobit species is a human Hobit species wherein at least one and preferably at least two and more preferably at least 4 of the zinc fingers are substituted by the corresponding zinc finger or zinc fingers of a Blimp-1 protein. Preferably of a human Blimp-1 protein. Te invention thus further provides a human Hobit species wherein at least one zinc finger is replaced by another zinc finger, preferably from another zinc finger protein. In a preferred embodiment said another zinc finger protein is Blimp-1, preferably human Blimp-1.

Expression of a Hobit species in a cell is preferably elevated and/or enhanced by providing said cell with a nucleic acid comprising a coding region for said Hobit species. Expression of a Hobit species in a cell that expresses said Hobit species is preferably reduced by providing said cell with a nucleic acid comprising and/or encoding an anti-sense sequence for said Hobit species. Said nucleic acid is preferably provided to said cell by means of a gene delivery vehicle comprising said nucleic acid. In one embodiment the invention therefore provides a gene delivery vehicle comprising a nucleic acid encoding a Hobit species or a functional part, derivative and/or analogue thereof. Preferably said nucleic acid encodes human Hobit-L, Hobit-M, Hobit S and/or Hobit XS or a mammalian homologue thereof. In another embodiment the invention provides a gene delivery vehicle comprising a nucleic acid or functional equivalent thereof that is or codes for an anti-sequence to a sequence as depicted in FIGS. 8, 9 and/or 10. As mentioned herein above, there are several different types of anti-sense strategies developed at present. An antisense can be developed against the coding region, the mRNA or to the precursor RNA. The sequence of the precursor RNA is obtainable from the genomic sequence as depicted in FIG. 11. An antisense sequence preferably comprises a stretch of between 18 to 40 nucleotides. In a preferred embodiment said gene delivery vehicle comprises a virus particle and/or a liposome. Preferably said virus particle is a retrovirus particle, preferably a lentivirus particle, an adenovirus particle and/or an adeno-associated virus particle.

As many of the viral gene delivery vehicles require the packaged nucleic acid to at least comprise the viral packaging signal and/or a viral repeat sequence the invention further provides an isolated and/or recombinant nucleic acid of the invention, further comprising at least one virus specific sequence preferably a packaging signal and/or a virus repeat sequence. In another embodiment a nucleic acid as provided by the invention comprises at least one coding region for a virus. Preferably said packaging signal, virus repeat sequence and/or coding region is of a retrovirus, preferably a lentivirus, an adenovirus and/or an adeno-associated virus. Further provided is an isolated and/or recombinant nucleic acid according to the invention encoding a Hobit species wherein a zinc finger as identified in FIG. 1, is replaced by an artificial zinc finger and/or a zinc finger of a different Hobit species.

The invention further provides the use of a nucleic acid according to the invention, for altering the activation and/or differentiation state of a Killer-cell provided therewith. The invention further provides the use of a nucleic acid according to the invention, for the preparation of a vaccine. Also provided is the use of a nucleic acid according to the invention for the preparation of a medicament for altering a Killer-cell mediated immune response in an individual.

In another aspect the invention provides a method for determining the status of the Killer-cell specific immunity in an individual at the time a Killer-cell containing sample was obtained from said individual said method comprising determining in Killer-cells of said sample the expression of a Hobit species. Preferably said method further comprises comparing said expression with a reference.

### BRIEF DESCRIPTION OF THE DRAWINGS

FIG. 1

Homology of Hobit and Blimp-1ZNF domain.

FIG. 2

Schematic representation of Hobit and Blimp-1

FIG. 3

Hobit upregulation in the presence of IL-15

FIG. 4

Hobit upregulation in treated CD+8 cells and NK cells

FIG. 5

Increase in IFNg production in treated cells

FIG. 6

Differential gene expression in PMA/ionomycine stimulated YT2C cells after introduction of a hobit species by lentiviral transduction.

FIG. 7

Putative molecular cascade of Hobit signaling.

FIG. 8

Nucl and aa sequences of splice variants of Hobit (ZNF683).

FIG. 9

Alignment of Hobit species on nucleotide and amino acid level.

FIG. 10

Alignment of Human and mouse Hobit.

FIG. 11

Genomic region sequence of Hobit

### EXAMPLES

### Sequence of Hobit

Hobit is annotated as ZNF683. By cloning cDNA from NK cells, we found multiple splice variants of Hobit. We name the annotated form: Hobit-XL. We found several deletions: 20 nucleotides (delta 20), 60 nuceotides (delta 60), 66 nt (delta 66) and 207 nucleotides (delta 207). Up till now, we found several combinations with the following combination of deletions:

| Splicevariants | | | aminoacids | nucleotide |
|---|---|---|---|---|
| No deletions: | Extra large | Hobit-XL | 509 | 1530 |
| delta 60 alone | Large | Hobit-L | 489 | 1470 |
| delta 60 + | Medium | Hobit-M | 467 | 1404 |
| delta 66 | | | | |
| delta 20 + | Small | Hobit-S | 369 | 1110 |
| delta 60 | | | | |
| Delta 20 + | Extra | Hobit-XS | 300 | 903 |
| delta 60 + | small | | | |
| delta 207 | | | | |

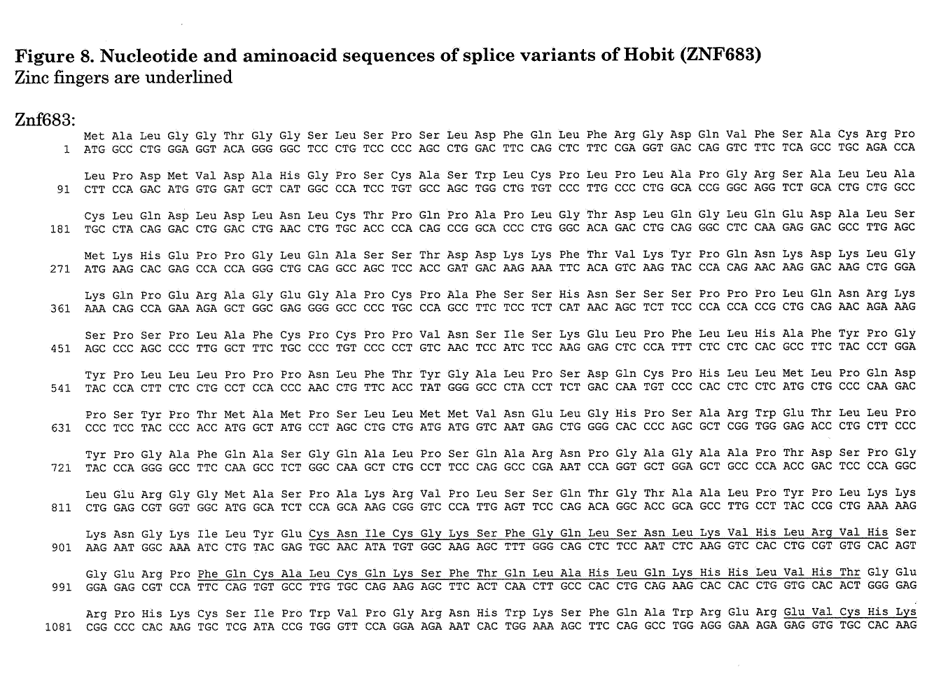

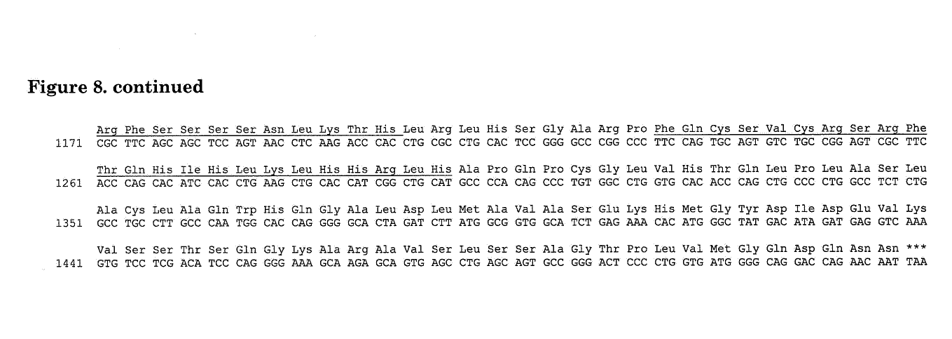

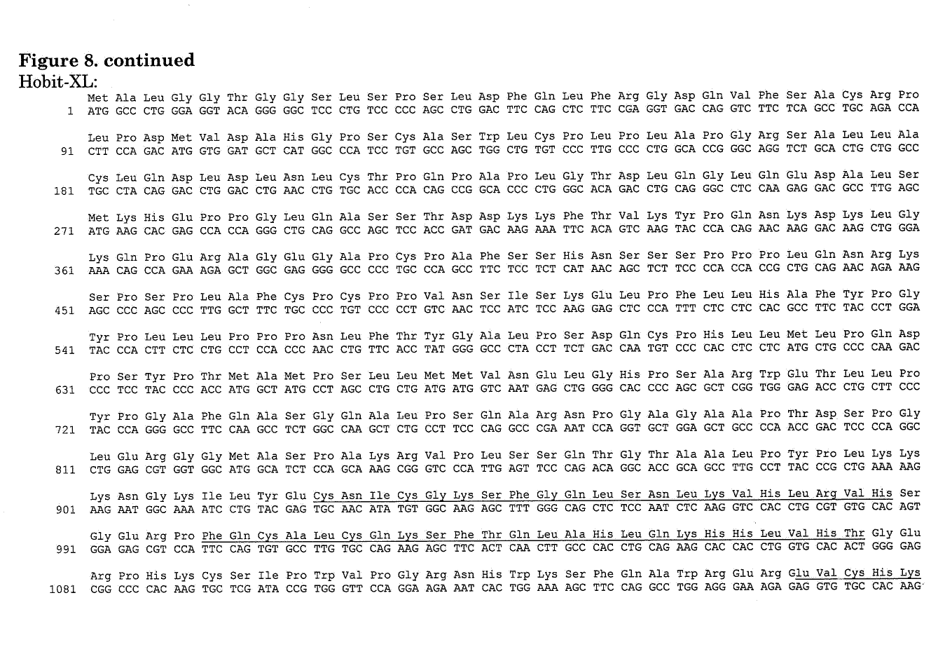

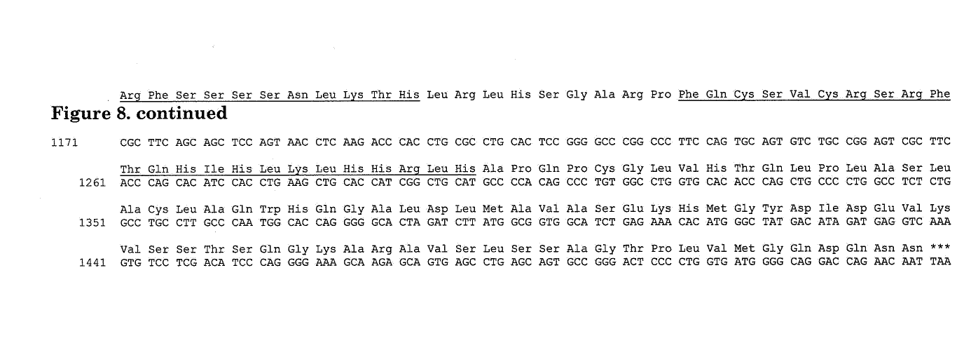

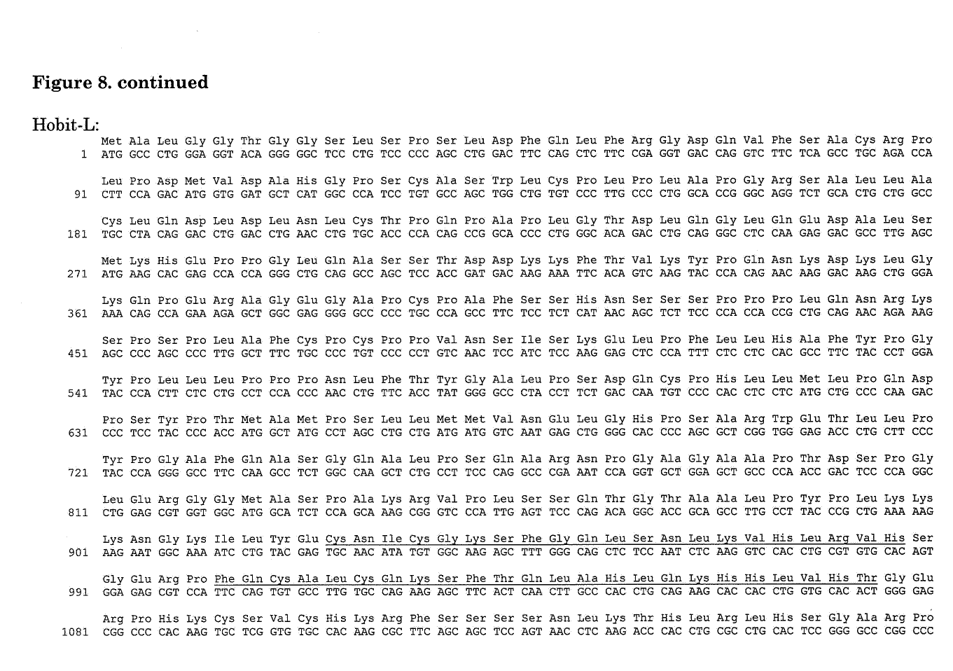

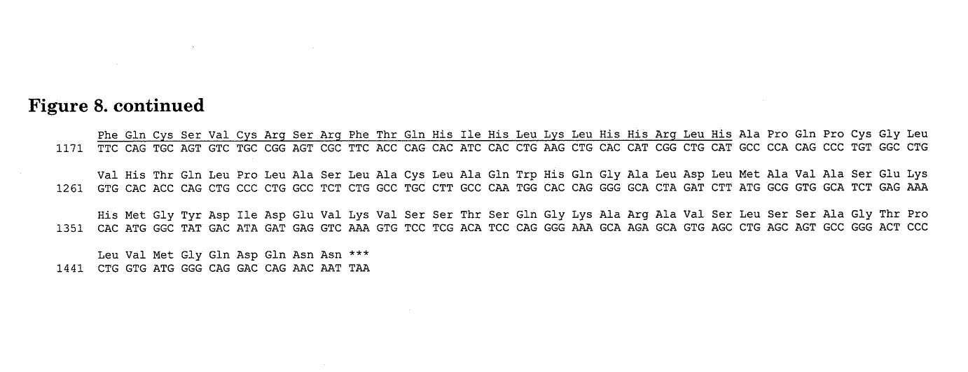

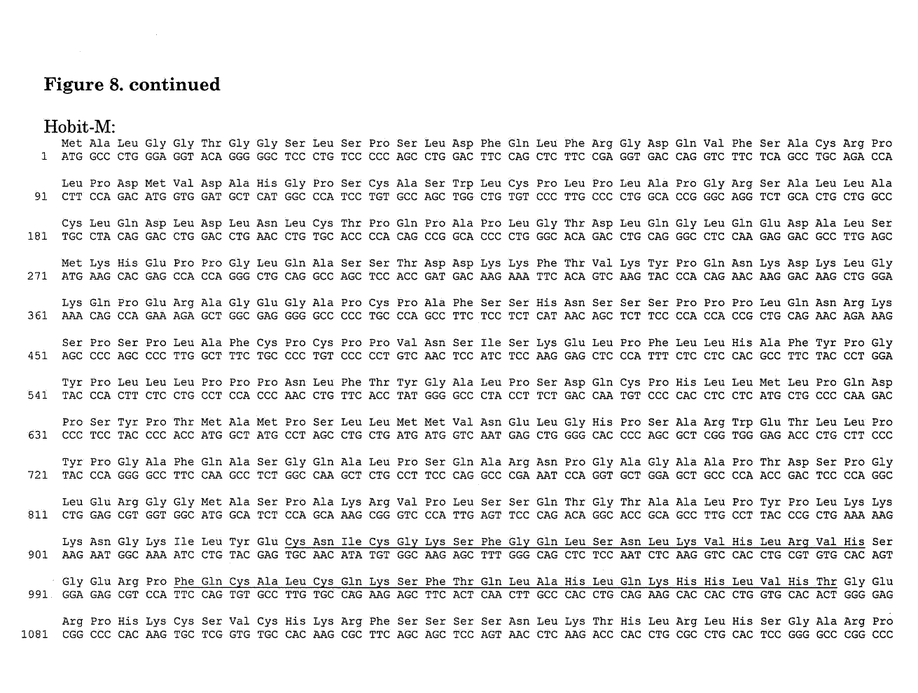

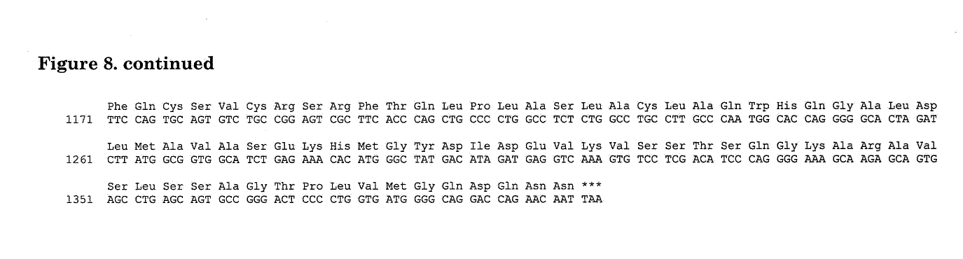

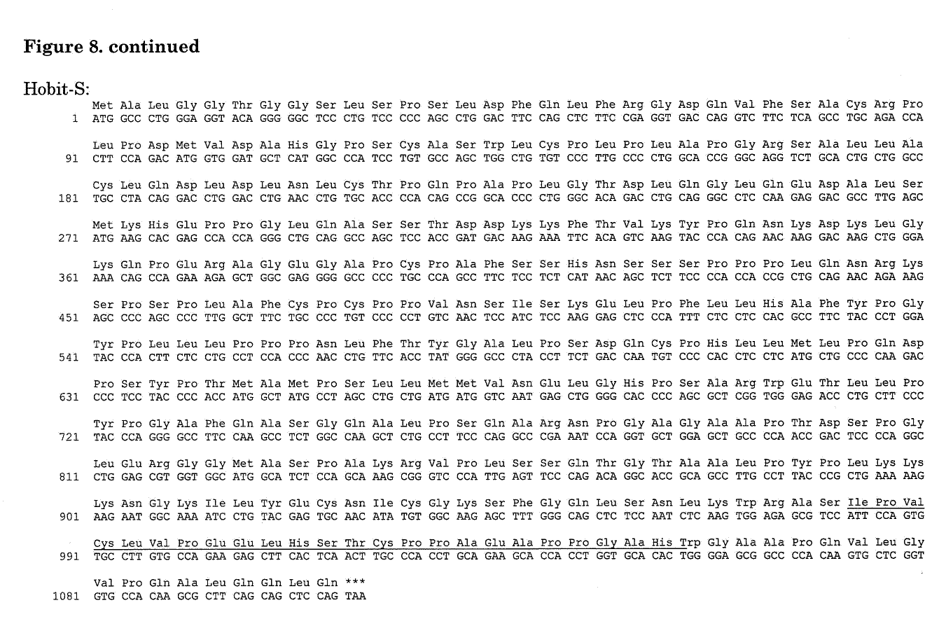

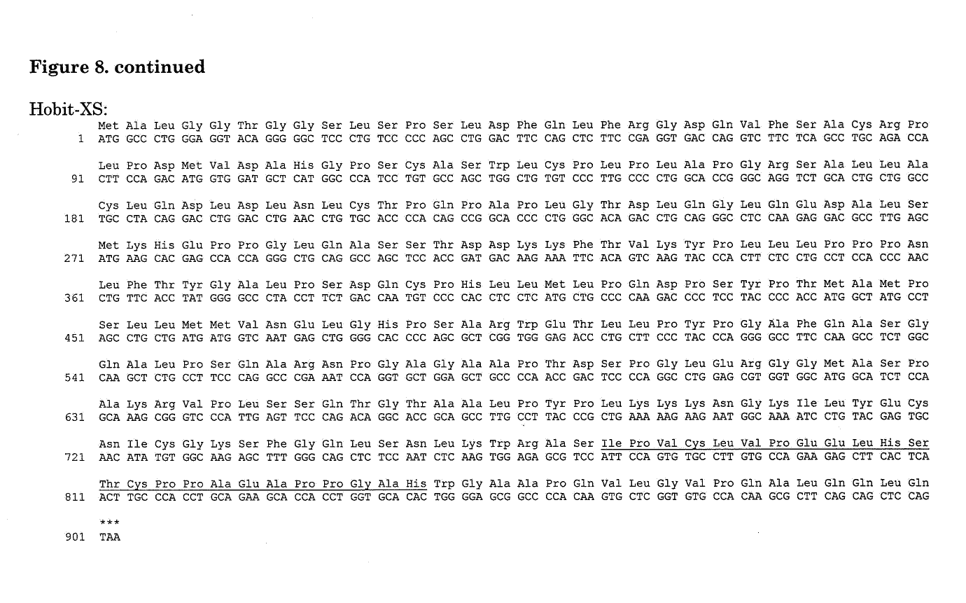

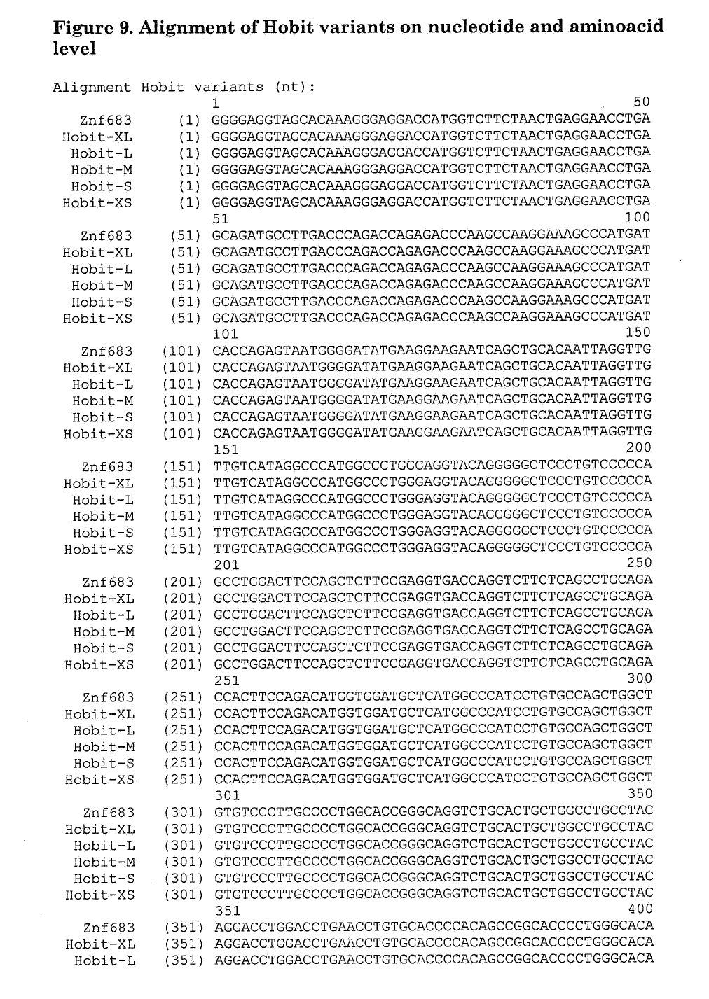

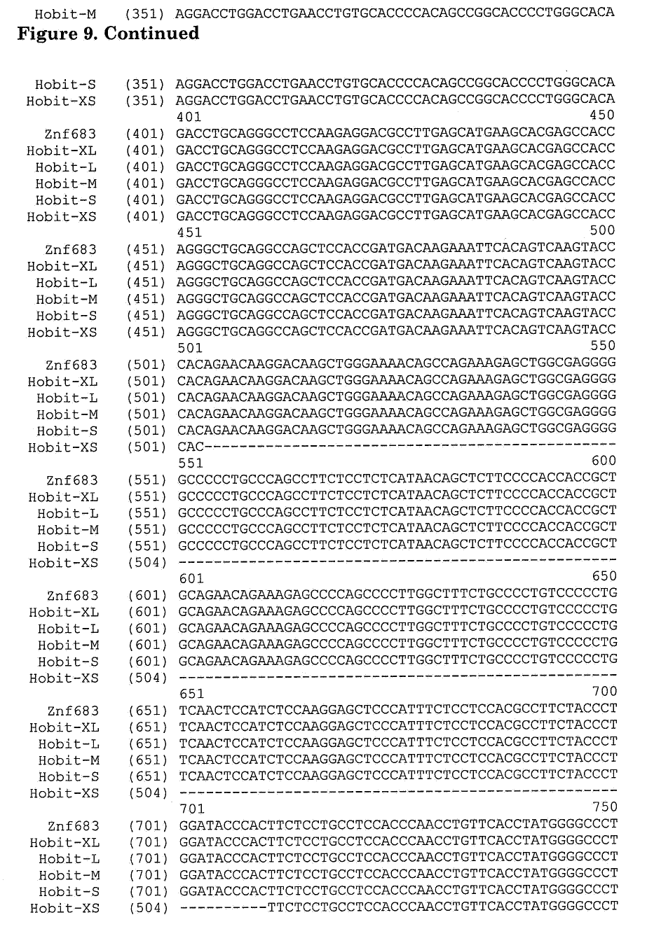

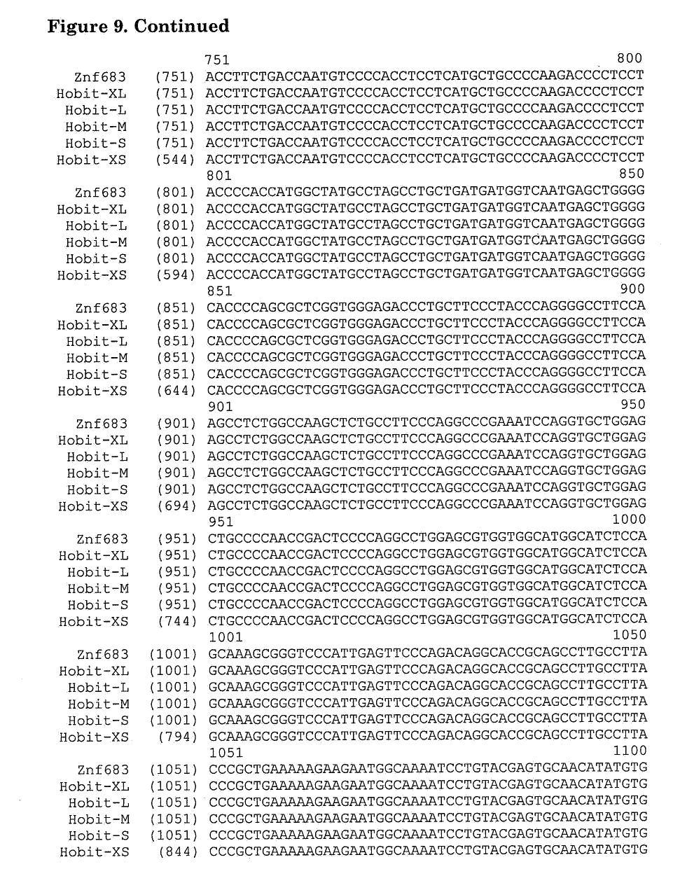

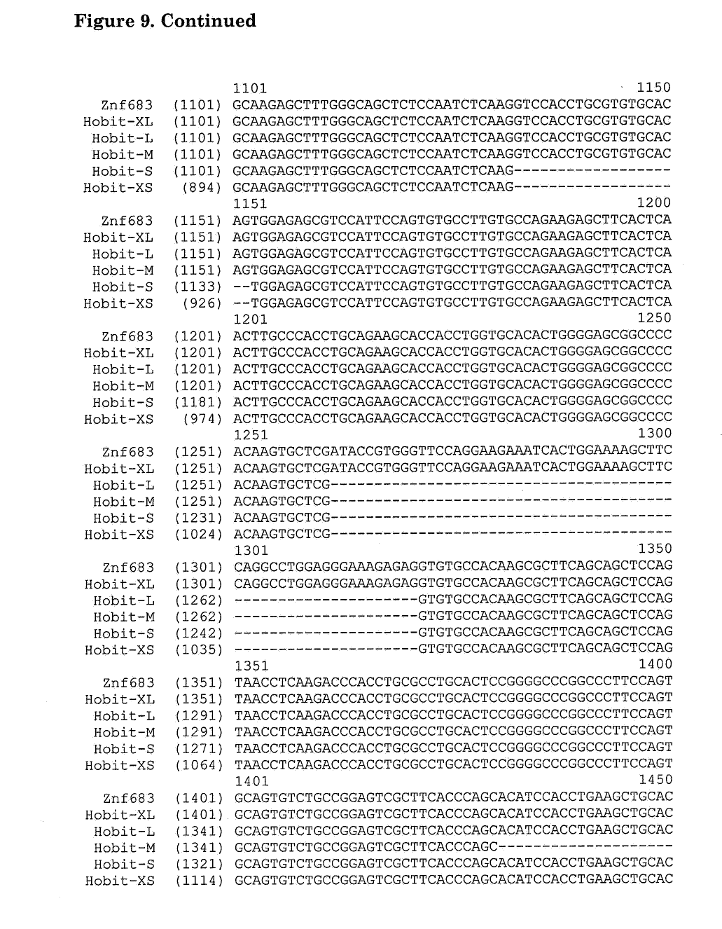

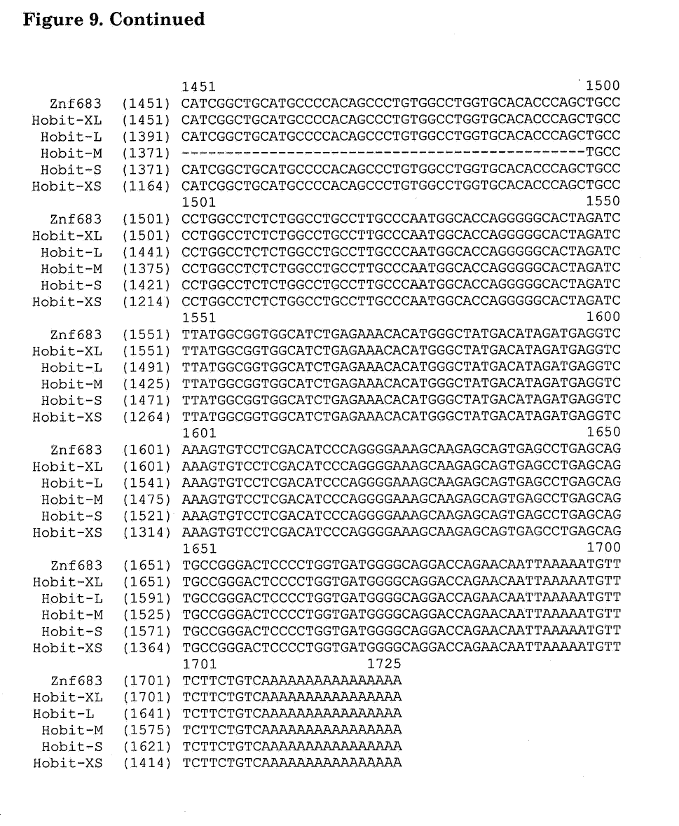

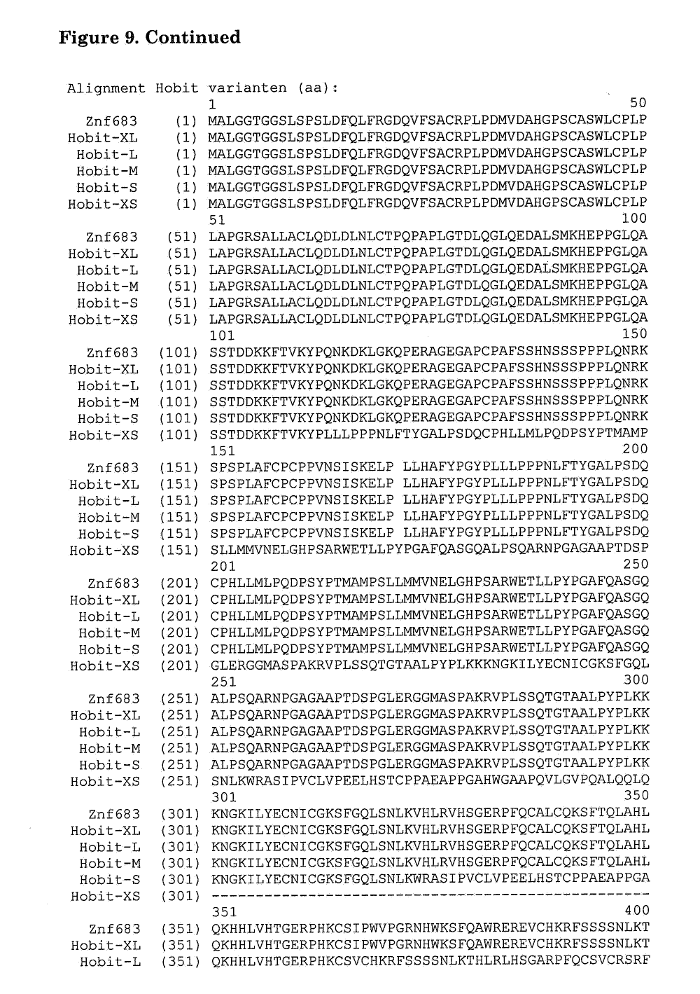

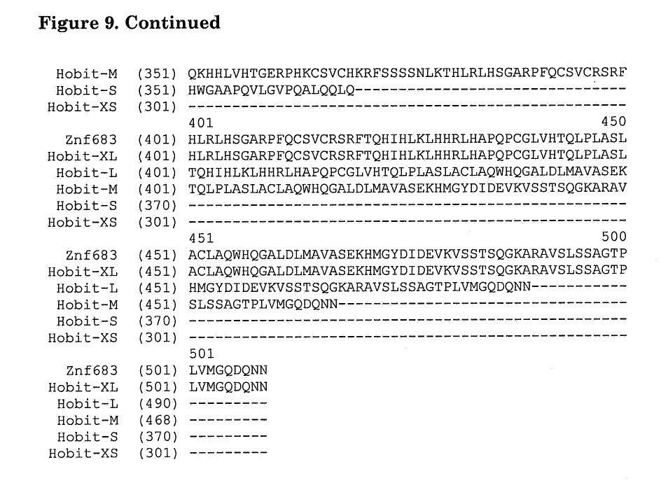

In FIG. 8, you can find the nucleotide sequence with its translation of all splicevariants. The annotated sequence (which is similar to Hobit-XL) is named ZNF683. In FIG. 9, all variants are aligned on the nucleotide and aminoacid levels.

In CD8 effector-memory, CD8 memory cells, NK cells and in lung tissue we found both Hobit-XL and Hobit-L in a ratio of ˜30%/70%. In testis, we found a different ratio; 50/50. In 3 large granular lymphocyte (LGL disease) samples we found ratio's of Hobit XL/L; in #1: 40/60, in #2: 30/70 in #3: 50/50. In none of the tested samples we found Hobit-S. We did not yet examine Hobit-M and Hobit-XS.

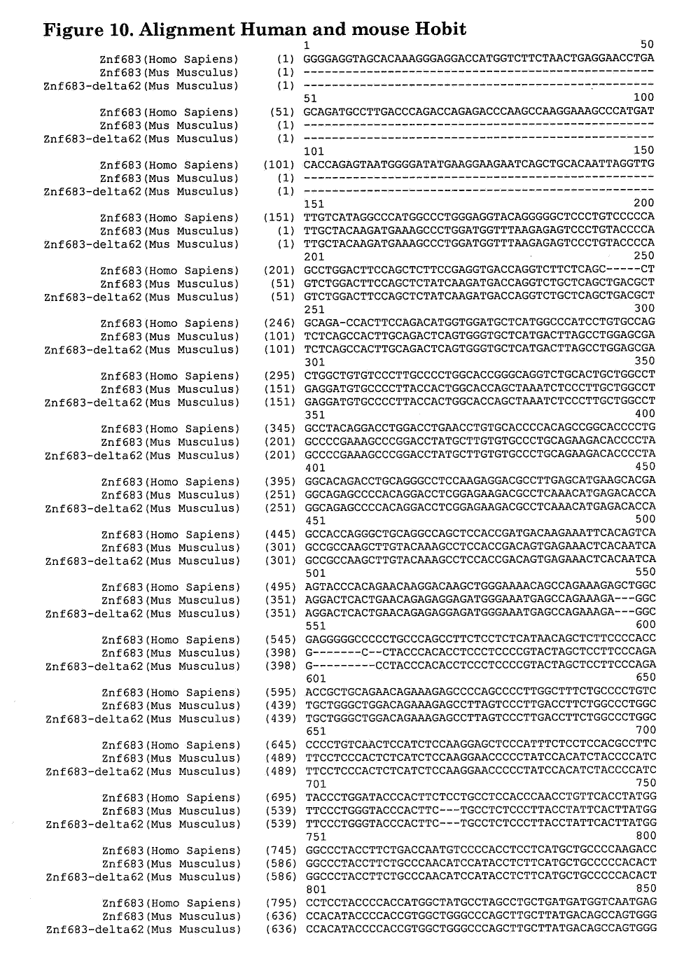

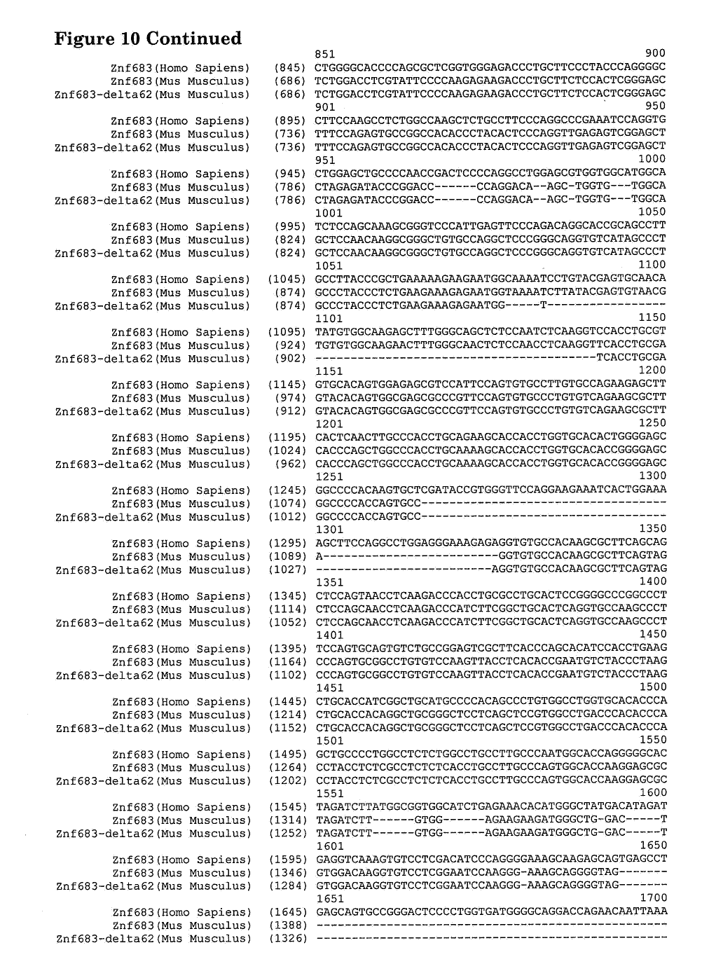

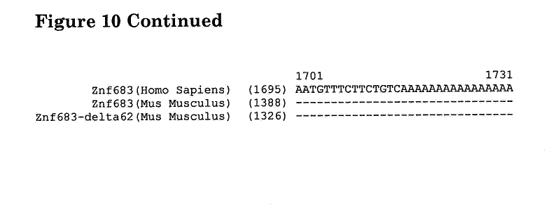

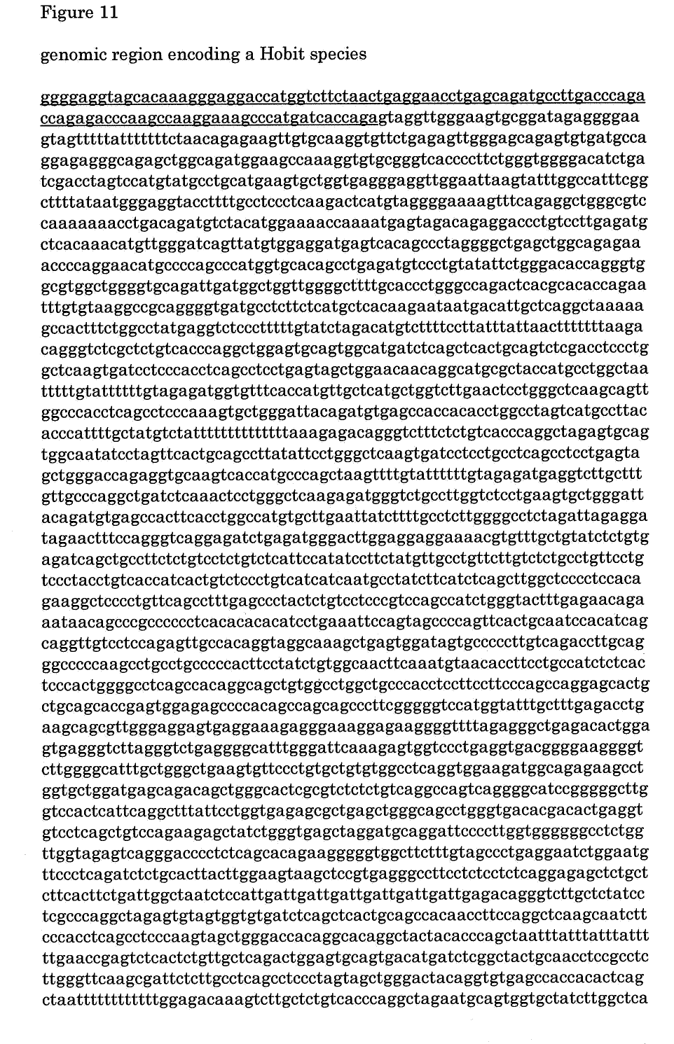

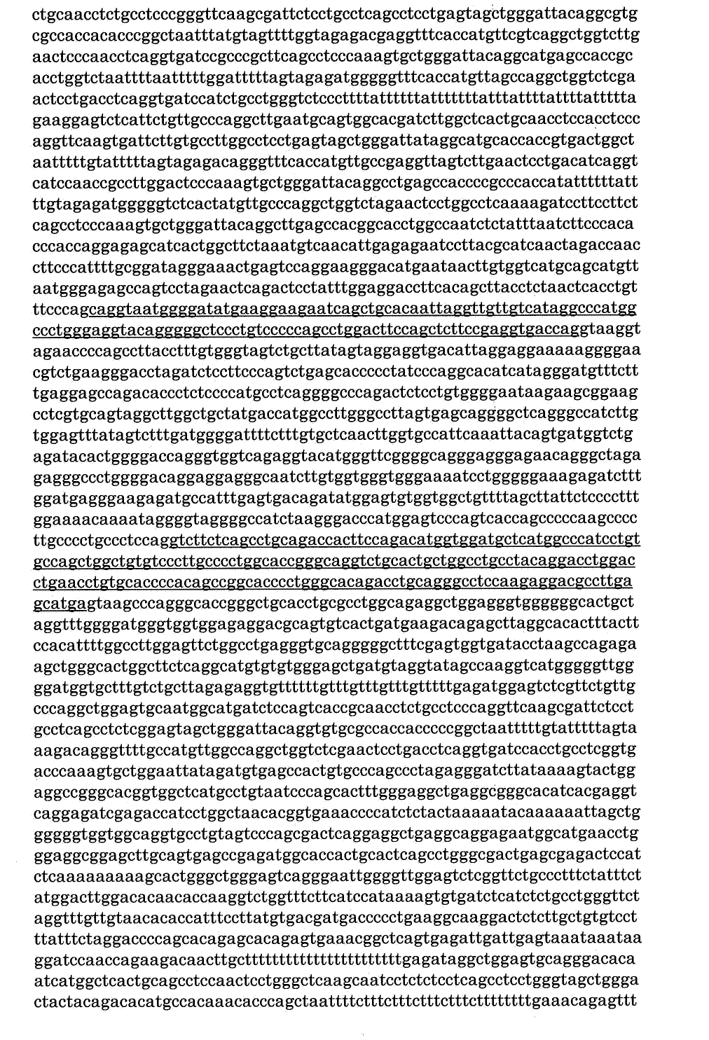

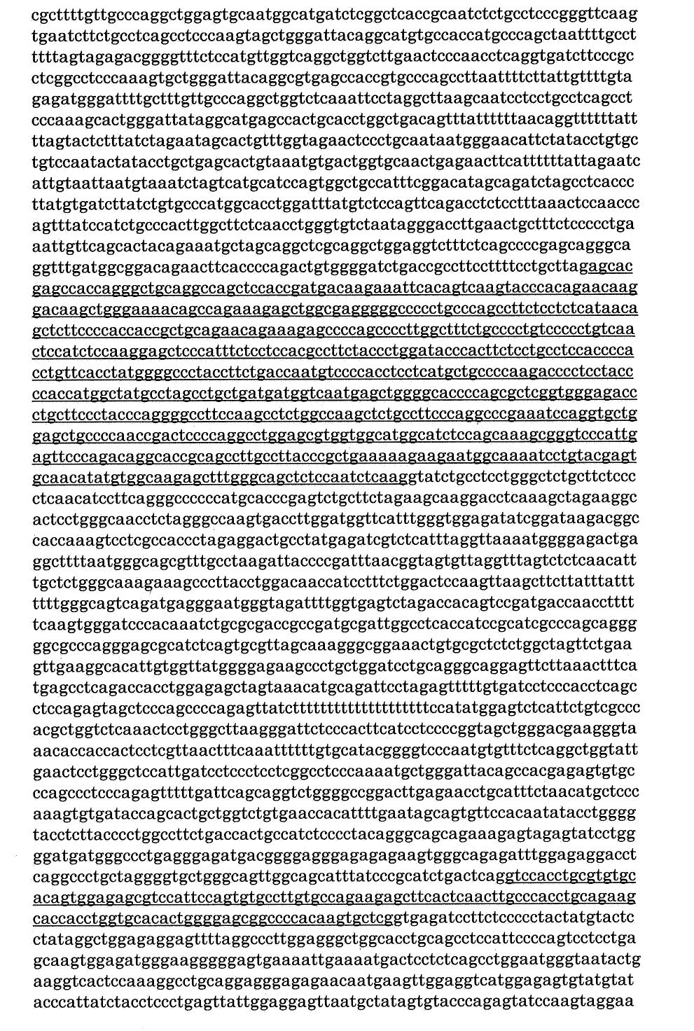

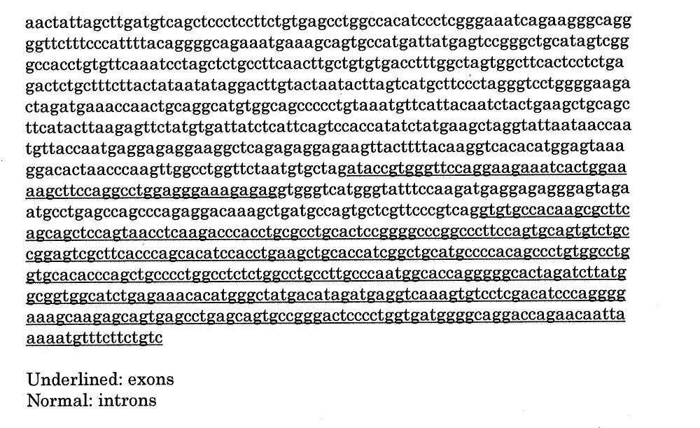

In FIG. 10, the alignment between human and mouse is depicted. We also found a smaller splicevariant that has a deletion of 62 nucleotides. These are approximately 65% homologous with human. The new genbank database indicates that human ZNF683 does not have a murine homologue. The murine sequences that we use to align with human ZNF683 is the sequence that we obtained from an effector-memory CD8+ T cell subpopulation (CD62L-/CD44+) from a mouse spleen.

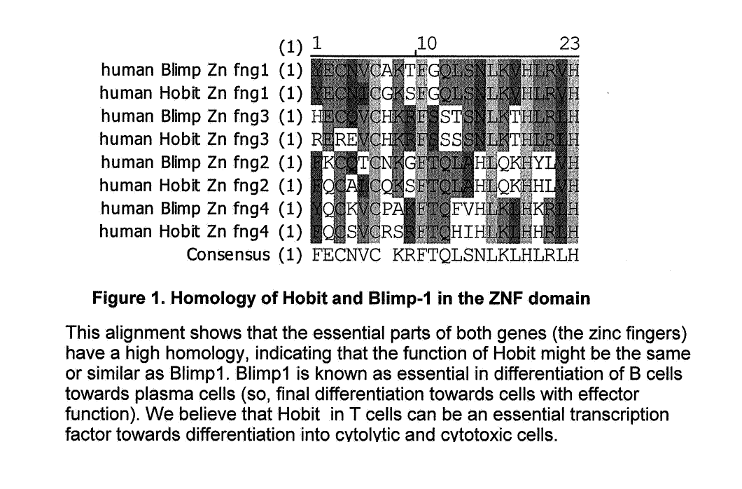

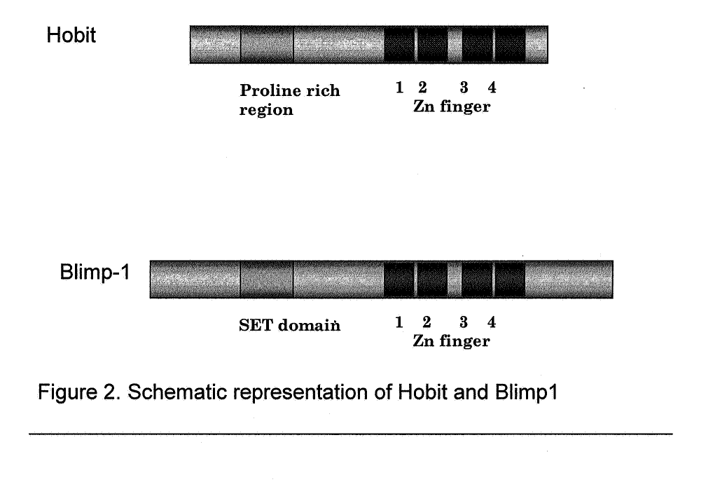

Homology between PRDM1 (Blimp-1) and ZNF683 (Hobit) lies in the zinc finger domain (FIG. 1). A simplified scheme of the essential domains present in Hobit and Blimp-1 are depicted in FIG. 2. In Hobit and Blimp1, the zinc finger domain consists of 4 zinc fingers of the C2H2 type. On the 5′ site of the zinc finger domain, Hobit has a proline rich domain important for protein-protein interactions. In Blimp-1, there is a SET domain, also important for protein-protein interactions.

### Differential Expression of Hobit

Differential expression of Hobit was found in a microarray experiment using 3 distinct CD8+ subsets (Table 1a) and in an experiment using CMV specific CD8+ T cells during a primary CMV infection (Table 1b). (M&M: microarray experiments).

### Induction of Hobit:

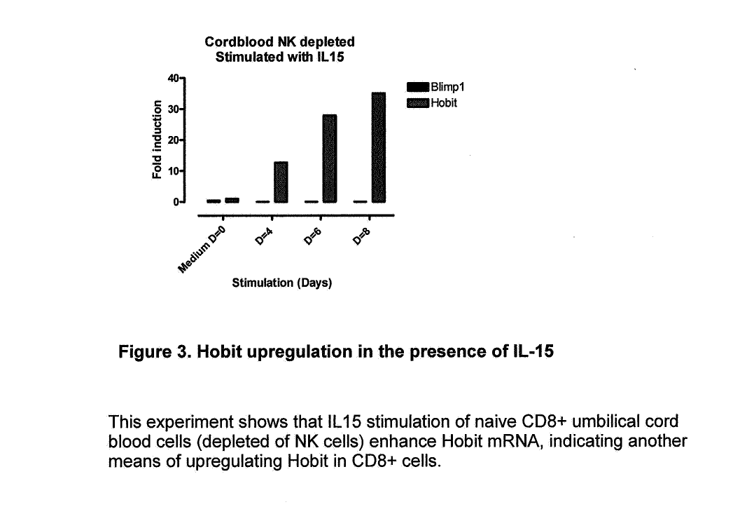

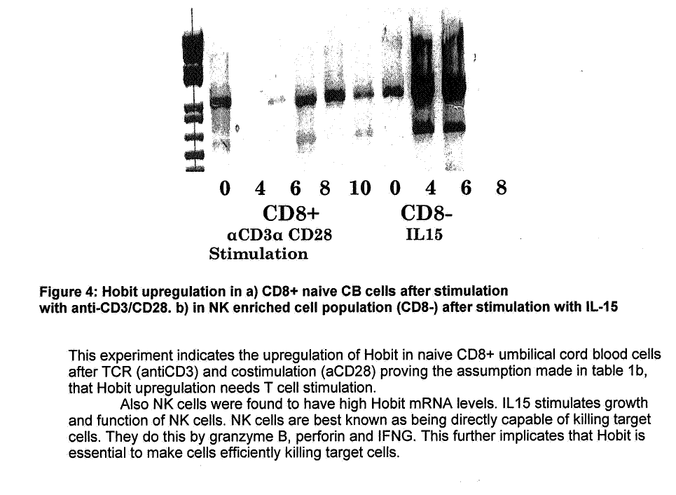

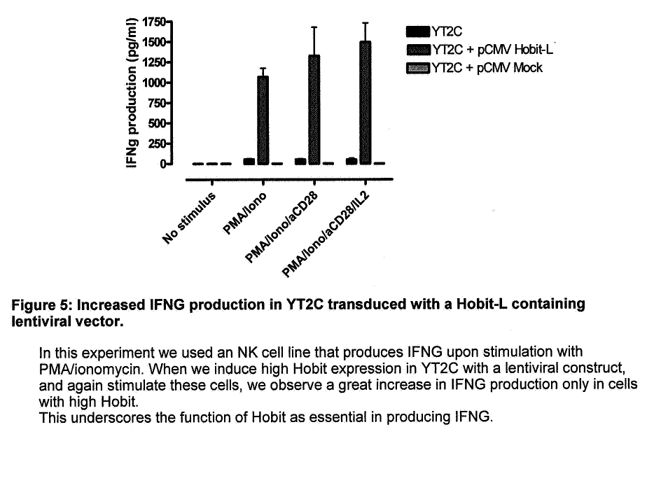

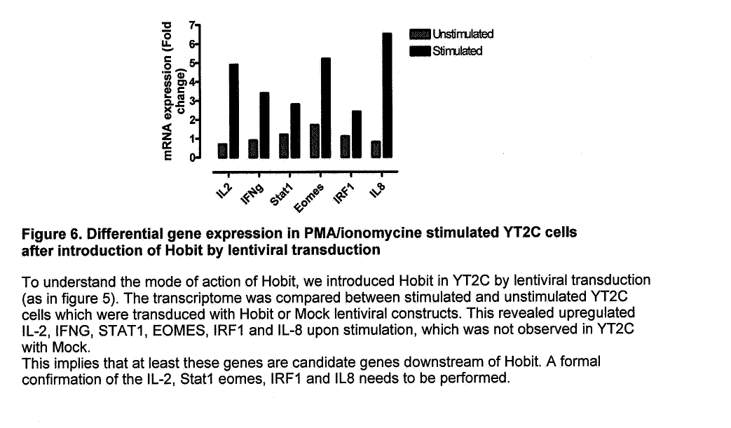

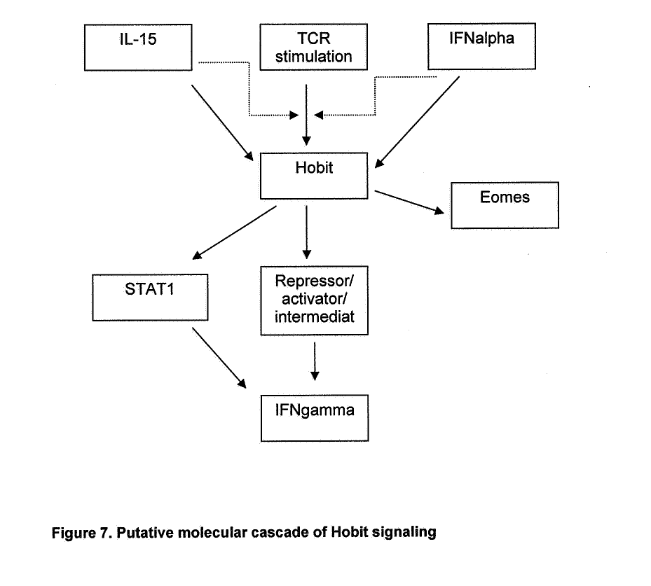

We know that Hobit can be induced
in CD8+ naïve cord blood cells cultured in the presence of IL-15. (M&M: IL-15) (FIG. 3)
in CD8+ naïve cord blood cells after stimulation with anti-CD3 moab in combination with anti-CD28 moab: (M&M: aCD3/aCD28) (FIG. 4a) (this experiment has only been performed once and need to be repeated!)
in NK cells after stimulation with IL-15 (10 ng/ml) (FIG. 4b) (M&M: Standard PCR of Hobit)
   Overexpression of Hobit (M&M: cloning of Hobit-L and M&M: Lentiviral transduction of YT2C)
augments IFNG production in YT2C (NK cell line) after stimulation with PMA/ionomycine in combination with anti-CD28 moab (FIG. 5)

### M&M: Microarray Experiments

### Cell Isolation of Cd8+ Subsets

Cells were isolated from buffy coats by a two step procedure. After Ficoll, CD8+ cells were isolated by CD8+ microbeads (Miltenyi Biotec, Utrecht, the Netherlands) and stored overnight at 4° C. in serum containing medium. CD8+ cells were then stained with CD27-FITC (7C9, home-made), CD45RA-RD1 (Coulter, Miami, L) and CD8-APC (BD Pharmingen, San Diego, Calif.) and FACS sorted using a FACS Vantage (Beckton Dickinson (BD), San Diego, Calif.) in naïve CD8+ cells (CD8+CD45RA^{high}CD27^{high}), effector CD8+ cells (CD8+CD45RA^{high}CD27^{low}) and memory CD8+ cells (CD8+CD45RA^{low}CD27^{high}).

### Cell Isolation of Cd8+ Effector Cells During Primary CMV Infection

PBMCs from renal transplant patients were thawed and either FACS sorted (peak CMV) or isolated using the MACS beads isolation procedure (Miltenyi Biotec, Utrecht, the Netherlands) using anti-APC beads. Cells isolated 2 weeks after CMV peak were stained with HLA-DR-FITC (BD Biosciences), CD38-PE (BD Biosciences) and CD8-APC (BD Pharmingen) and HLA-DR^{high}CD38^{high}CD8⁺ were sorted using FACS Vantage (Beckton Dickinson, San Diego, Calif.). CMV specific cells from one year after transplantation (long term) were stained with APC conjugated tetramers loaded with pp65. After staining, tetramer+ cells were isolated by APC microbeads (Miltenyi Biotec, Utrecht, the Netherlands).

RNA isolation Total RNA was isolated from cells with the nucleospin RNA isolation kit (Machery-Nagel, Duren, Germany) according to manufacture's instructions. RNA Amplification, Labelling and Hybridization was performed at Service XS (Leiden, The Netherlands) with Whole human genome two-color arrays from Agilent.

### M&M Isolation and Purification of Cells

Human peripheral blood mononuclear cells (PBMCs) from buffy coats of healthy donors were isolated by Ficoll-Isopaque density gradient centrifugation (Nycomed; Pharma, Oslo, Norway). Umbilical cord blood mononuclear cells (UCBMCs) were used to obtain naive T cells. The study was approved by the local medical ethics committee of the Academic Medical Center.

CD8+ T cells and CD16+NK cells were purified either from total PBMCs or UCBMCs by selection using the Dynal bead selection system and the MACS system. Brie Ly, PBMCs were stained with moabs against CD16 for 30 minutes and washed with PBA. Goat anti mouse immunoglobulins dynal beads were then incubated for 30 minutes. The Dynal magnet was applied according to the manufacturer's instruction. The CD16+ NK cells were then stored on ice for further use. The NK depleted cell population was then stained with CD8 microbeads for 15 minutes at 8° C. After washing, cells were resuspended in incubation buffer and enrichment was performed with the VarioMACS magnet according to manufacturer's instructions. The sample purity was assessed by Luorescence-activated cell sorter (FACS) with APC-conjugated CD8 and PerCP-conjugated CD3 mAbs (purity, >95% CD3+ CD8+).

### M&M IL-15

UCBMCs, or purified CD8+ T-cell subsets, were cultured for 7 days at 37° C. in 5% CO2 atmosphere, in culture medium in the presence or absence of 10 ng/ml IL-15 (R&D Systems).

### M&M: Standard PCR of Hobit:

Primer pairs for all variants of Hobit in a standard PCR reaction: {Hobit seq mid. Fwd}: CACCGCTGCAGAACAGAAAG & {Hobit2.0 CLN Revs} GCAAGAGCAGTGAGCTG. This amplifies a product of approximately 1100 nt.

### M&M Intracellular Detection of IFNG

The cytokine-producing capacity was assessed after phorbol myristate acetate (PMA; 2 ng/mL) and ionomycin (1 μM/mL) stimulation in the presence of brefeldin A (1 μM; all from Sigma Chemical, St Louis, Mo.) for 4 hours. Cells were fixed in 2% paraformaldehyde, permeabilized with PBS containing 0.5% (wt/vol) bovine serum albumin (BSA), and 0.5% saponin followed by staining with PE-labeled anti-IFN-γ and a control mAb (gamma-1).

Cell Culture of YT2C

YT2C is a subline of the YT cell line, originally established from a child with acute lymphoblastic lymphoma and thymoma [1]. Cell lines were cultured in Iscove's modified Dulbecco's medium (IMDM; Invitrogen), supplemented with 10% (v/v) heat-inactivated FCS (ICN Biomedicals GmbH, Meckenheim, Germany), 100 U/ml penicillin and 100 μg/ml streptomycin.

293T cells were cultured in Dulbecco's modified Eagle's medium (DMEM) containing 10% (v/v) heat-inactivated FCS (ICN Biomedicals GmbH, Meckenheim, Germany), 100 U/ml penicillin and 100 μg/ml streptomycin.

Cloning of Hobit-L

To obtain the Hobit-L gene, the 1467 bp fragment was amplified by PCR with the following primers: GGTACCTCGCCCTGGAGGTACAGGG (Hobit4.0 CLN Forw.) and GCAAGAGCAGTGAGCTG (Hobit2.0 CLN Revs). The gene without the start codon was sequenced and cloned as a Xho1/EcoR1 fragment into pMT2SM-HA which added a HA tag in front of the open reading frame.

Lentiviral Vector Construction and Infection

To generate the Hobit Large lentiviral construct, restriction enzymes PstI and EcoR1 were used to subclone the HA-tagged Hobit sequence into the lentiviral vector backbone RRL-cPPT-CMV-X2-PRE-SIN-IRES-eGFP (CMV). The lentiviral vector pRRLcmvgfpsin contains a phosphoglycerate kinase or cytomegalovirus promoter driving green Luorescent protein (GFP) expression. The HA-tagged Hobit Full length gene was cloned before the GFP open reading frame which yielded the new vector pRRLcmvgfpsinHobit-L (Hobit-L).

The vector pCMVHobit-L and the backbone without Hobit construct (pCMV Mock) were used in the third generation self inactivating lentiviral vector system (2, 3). Brie Ly, expression vectors for HIV Gag/Pol, HIV Rev, VSVg and the viral transfer vector were cotransfected overnight using calcium phosphate into 293T cells. The medium was changed and virus was collected twice for 24 h, passed through 0.45 μm filters and stored at −80° C.

Cells were seeded in 24 well plates at a density of 106 cells/well and incubated with virus for 4 h in the presence of 8 μg/ml polybrene and spinfection for 2 hours at 2400 rpm for 2 hours. The cells were harvested 3 days after transduction and GFP-positive cells were sorted using a FACS-Aria (BD Biosciences) cell sorter. By culturing YT2C cells tranfected with either pCMVHobit-L or pCMVMock at 1 cell per well the clone was selected with the highest GFP expression measured by Luorescent-activated cell sorting (FACS).

To assess whether overexpression of the Hobit gene had occurred, cell line YT2C was transfected with Lentivirus as described before. RNA was isolated (Machery-Nagel NucleoSpin RNA II kit) and reverse transcribed to obtain cDNA. Hobit qPCR was performed using primers CATATGTGGCAAGAGCTTTGG (Hobit RT 9.0 Forw.) and GGCAAGTTGAGTGAAGCTCT (Hobit RT 9.0 Revs.).

In vitro Stimulation

The cytokine-producing capacity was assessed after phorbol myristate acetate (PMA; 2 ng/mL) and ionomycin (1 μM/mL)) stimulation (1 μM; all from Sigma Chemical, St Louis, Mo.) in the presence or absence of aCD28 (3 ug/ml) and IL2 (50 Units/ml). After 4 hours supernatants were collected and the IFN_{Y} production was measured by for IFN_{Y} enzyme-linked immunosorbent assay (ELISA; Sanquin, Amsterdam, the Netherlands).

Microarray

YT2C cells transfected with either pCMVHobitL or pCMV Mock were stimulated with or without PMA, Ionomycin, aCD28 as described earlier. RNA was extracted as before. Protocol micro-array

Illumina microarray expression results are given in arbitrary units from Hobit-L transfected samples were divided by the numbers generated by the mock transfected YT2C cell line. The ratio between these samples is plotted in FIG. 6.

### REFERENCES

1. Yodoi J, Teshigawara K, Nikaido T et al. TCGF (IL 2)-receptor inducing factor(s). I. Regulation of IL 2 receptor on a natural killer-like cell line (YT cells). J Immunol 1985; 134: 1623-30.
2. R. Zufferey, T. Dull, R. J. Mandel, A. Bukovsky, D. Quiroz, L. Naldini et al., Self-inactivating lentivirus vector for safe and efficient in vivo gene delivery. J Virol 1998; 72: 9873-9880.
3. T. Dull, R. Zufferey, M. Kelly, R. J. Mandel, M. Nguyen, D. Trono et al., A third-generation lentivirus vector with a conditional packaging system. J Virol 1998; 72: 8463-8471.

TABLE 1a
Hobit mRNA in CD8+ subsets

| | |
|---|---|
| Hobit in CD8+ subsets | Hobit expression |
| | (no distinction |
| | between splicevariants) |
| Subset of cells | Fold change |
| | Expression value of indicated |
| | subset of cellsdivided by the |
| | expression value of naive |
| | CD8+ cells |
| Naïve CD8+ cells | 1.0 |
| (CD8+CD45RA+CD27+) | |
| Effector-memory CD8+ | 17.75 |
| (CD45RA+CD27−CD8+) | |
| Memory-type CD8+ | 4.66 |
| (CD45RA−CD27+CD8+) | |

Effector-memory CD8+ cells have a high cytolytic capacity. This is evident by a high granzyme B, Perforin and IFNG content. These cells can react very quickly. These traits are opposed to the memory-type CD8+ cells that need several days to get activated (high granzymeB, Perforin and IFNG). The high Hobit mRNA expression in the CD8+ effector-memory cells indicates that Hobit is involved in the phenotype of these effector memory CD8+ cells.

TABLE 1b
Hobit mRNA in primary CMV infection

| Hobit in primary | Hobit |
|---|---|
| CMV infection | (no distinction between |
| | splicevariants) |
| Subset of cells | Fold change |
| | Expression value of |
| | indicated subset of |
| | cellsdivided by the |
| | expression value of naive |
| | CD8+ cells |
| Naïve CD8+ cells (CD8+CD45RA+CD27+) | 1.10 |
| Peak of viral infection | 11.94 |
| (CD8+HLA−DR+CD38+) | |
| 1 year after infection (CMV specific | 10.10 |
| cells: Tetramer pp65+ CD8+ | |
| Latency: ~20 years after infection: | 12.06 |
| Tetrame pp65+ CD8+ | |

This experiment shows that during an acute primary infection of CMV (Cytomegalovirus), CD8+ cells specifically reacting against CMV have high Hobit mRNA. We know that the reacting T cells are activated via TCR (CD3) and costimulation (CD28), indicating that signaling via TCR is at least one means of Hobit upregulation.

1. A method for altering activation and/or differentiation of Killer cells comprising altering the expression of a Hobit species in said Killer-cells.

2. A method according to claim 1, wherein said activation and/or differentiation is elevated through increasing expression of said Hobit species in said Killer-cells.

3. A method according to claim 1, wherein said activation and/or differentiation is reduced through decreasing expression of said Hobit species in said Killer-cell.

4. A method according to any one of claims 1-3, wherein a Killer-cell or a Killer-cell precursor of said Killer-cell is provided with a nucleic acid for altering the expression said Hobit species in said Killer-cell or a progeny Killer-cell of said precursor.

5. A method according to claim 4, wherein said nucleic acid is provided to a naive T-cell.

6. A method according to any one of claims 1-5, wherein said Killer-cell or precursor thereof is a human Killer-cell or a human precursor thereof.

7. A method according to any one of claims 2, 4-7 wherein said expression is increased in cells in vitro.

8. A method according to claim 7, wherein said cells are a graft for transplantation comprising said Killer-cells or precursors thereof.

9. A method according to any one of claims 1-8, for enhancing a Killer-cell response of said Killer-cells or progeny thereof.

10. A method according to claim 9, for enhancing an antiviral response and/or an anti-tumour response of said Killer-cells.

11. A method according to any one of claims 1-10, for enhancing an antigen specific T-cell response, preferably an antigen-specific T-cell response specific for a tumour antigen or a virus antigen.

12. A method according to any one of claims 2, 4-11, wherein said elevating activation and/or differentiation comprises increasing interferon-gamma expression of said T-cells, increasing granzymeB or perforin expression in NK cells or another activation/differentiation marker for a Killer cell.

13. A method according to any one of claims 1-12, wherein said Killer-cells are memory T-cells, NK cells, effector T-cells, macrophages and/or CD28-CD27-CD4 positive T-cells.

14. An isolated and/or recombinant nucleic acid comprising a sequence as identified in FIGS. 8, 9 and/or 10, or a functional part, derivative and/or analogue thereof.

15. An isolated and/or recombinant nucleic acid according to claim 14, wherein said nucleic acid encodes a human Hobit-L, Hobit-M, Hobit S and/or Hobit XS or a mammalian homologue thereof.

16. A gene delivery vehicle comprising a nucleic acid encoding a Hobit species or a functional part, derivative and/or analogue thereof.

17. A gene delivery vehicle according to claim 16, wherein said nucleic acid encodes human Hobit-L, Hobit-M, Hobit S and/or Hobit XS or a mammalian homologue thereof.

18. A gene delivery vehicle comprising a nucleic acid or functional equivalent thereof that is or codes for an anti-sequence to a sequence as depicted in FIGS. 8, 9 and/or 10.

19. A gene delivery vehicle according to claim 17, wherein said anti-sense sequence comprises a stretch of between 18 to 40 nucleotides.

20. A gene delivery vehicle according to any one of claims 16-19, wherein said gene delivery vehicle comprises a virus particle and/or a liposome.

21. A gene delivery vehicle according to claim 20, wherein said virus particle is a retrovirus particle, preferably a lentivirus particle, an adenovirus particle and/or an adeno-associated virus particle.

22. An isolated and/or recombinant nucleic acid according to claim 14 or claim 15, further comprising at least one coding region for a virus, preferably a virus as mentioned in claim 21.

23. An isolated and/or recombinant nucleic acid according to claim 14, 15 or 22, encoding a Hobit species wherein a zinc finger of said Hobit species as identified in FIG. 1, is replaced by an artificial zinc finger and/or a zinc finger of a different Hobit species.

24. Use of a nucleic acid according to any one of claim 14, 15, 22 or 23, for altering the activation and/or differentiation state of a Killer-cell, preferably a T-cell, NK-cell or a macrophage, provided therewith.

25. Use of a nucleic acid according to any one of claim 14, 15, 22 or 23, for the preparation of a vaccine.

26. Use of a nucleic acid according to any one of claim 14, 15, 22 or 23, for the preparation of a medicament for altering a Killer-cell mediated immune response in an individual.

27. A method for determining the status of the Killer-cell specific immunity in an individual at the time a Killer-cell containing sample was obtained from said individual said method comprising determining in Killer-cells of said sample the expression of a Hobit species.

28. A method according to claim 27, further comprising comparing said expression with a reference.
